(19) United States
(12) Patent Application Publication
(10) Pub. No.: US 2024/0368549 A1
(43) Pub. Date: Nov. 7, 2024

(54) ENHANCEMENT OF HEMATOPOIETIC STEM CELL AND HEMATOPOIETIC PROGENITOR CELL EXPANSION WITH AGENTS THAT ACTIVATE TAM RECEPTORS

(71) Applicants: CANADIAN BLOOD SERVICES, Ottawa (CA); THE GOVERNING COUNCIL OF THE UNIVERSITY OF TORONTO, Toronto (CA)

(72) Inventors: Nicolas PINEAULT, Orleans (CA); Javed MANESIA, Burnaby (CA); Julie AUDET, Toronto (CA); Harinad MAGANTI, Toronto (CA)

(21) Appl. No.: 18/572,465

(22) PCT Filed: Jul. 22, 2022
(86) PCT No.: PCT/CA2022/051135

§ 371 Date: Dec. 20, 2023

Related U.S. Application Data
(60) Provisional application No. 63/224,941, filed on Jul. 23, 2021.

(51) Int. Cl. C12N 5/0789; A61K 35/28

(52) U.S. Cl. CPC C12N 5/0647; A61K 35/28; C12N 2501/125; C12N 2501/145; C12N 2501/26; C12N 2501/998; C12N 2501/999

(57) ABSTRACT

The present disclosure concerns the use of agents to increase the size of a population of normal hematopoietic stem and progenitor cells (HSPCs) outside of the body. The present disclosure also concerns the therapeutic use of a population of normal HSPCs, where the population size has been increased by the use of the agents. The present disclosure also concerns a kit that contains agents that can be use to increase the size of a population of normal HSPCs outside of the body.

### CROSS-REFERENCE TO RELATED APPLICATIONS AND DOCUMENTS

This is claiming priority on U.S. Ser. No. 63/224941 filed Jul. 23, 2022, the entire content of which is being incorporated by reference.

### TECHNOLOGICAL FIELD

The present disclosure pertains to compositions and methods for expanding populations of hematopoietic stem cells and hematopoietic progenitor cells in vitro or ex vivo.

### BACKGROUND

Hematopoietic stem and progenitor cells (HSPCs) comprise a small and heterogeneous pool of cells that are the precursors of all red blood cells (erythrocytes), platelets (thrombocytes) and major immune cells (leukocytes) in the body. HSPCs play an essential role in maintaining the homeostasis of the blood circulatory system in vertebrates though a regulated process termed hematopoiesis. Normally, most HSPCs are found in the bone marrow of vertebrates, the major site of hematopoiesis, and only small numbers of HSPCs are located in the peripheral blood. Hematopoietic stem cells (HSCs) are characterized by their unique capacity for self-renewal and multipotency. Conversely, hematopoietic progenitor cells (HPCs), which are formed from HSCs, have restricted lineage differentiation and lack the capacity to self-renew.

Hematopoietic stem cell transplantation (HSCT) has been used to re-establish bone marrow and immune function in patients with a variety of life-threatening malignant disorders (e.g, leukemia, lymphoma, multiple myeloma), including those whose malignancies are being treated with high dose chemotherapy or chemoradiotherapy, and non-malignant disorders (e.g., autoimmune diseases and hereditary diseases). There are two main types of HSCT: allogenic and autologous. Allogenic HSCT involves the transplantation of HSPCs to a patient from a healthy donor who is related or a donor who is not related but is an HLA-match for the patient. Autologous HSCT involves the extraction of HSPCs from the patient (generally prior to high dose chemotherapy or chemoradiotherapy) and their transfusion into the patient's bloodstream at a later date. Infection and graft-versus host disease are major complications of allogenic transplantation. While the risk of infection and graft rejection is reduced for autologous HSCT compared to allogenic HSCT, in cancer patients, it can increase the risk of relapse if the graft was inadvertently contaminated with cancer cells.

Graft sources for HSCT include bone marrow, peripheral blood or umbilical cord blood (UCB). HSPCs collected from different sources vary in cellular characteristics and clinical application. Despite having been used to treat a variety of diseases for the last several decades, HSCT utilizing bone marrow as a stem cell source has fallen out of favor as it suffers from the disadvantages of being inconvenient, uncomfortable and risky. Engraftment of peripheral HSPCs has become the most common HSCT practice due to quicker engraftment kinetics and ease of collection. However, harvesting HSPCs from peripheral blood requires first treating the donor with hematopoietic growth factors (e.g., granulocyte colony stimulating factor or G-CSF) in order to drive increase HSPC proliferation and mobilization from the bone marrow to the peripheral circulation. Such treatments can cause moderate to severe adverse effects in donors. Umbilical cord blood (UCB) is another important source of HSPCs used for HSCT. However, because the number of HSPCs in a single UCB graft is quite modest, HSCTs using UCB derived HSPCs are generally limited to pediatric patients.

The ability to expand and maintain functional HSPCs in vitro is critical to realizing the full potential of HSPC-based therapies. Various experimental approaches for expanding HSPCs in vitro have been developed including the use of cytokine cocktails, copper chelators, signaling molecules, stromal support, and even viral vectors. However, the development of therapies based on HSPCs expanded in vitro has been hampered by two major roadblocks: the lack of long-term engraftment and the loss of self-renewal in these cells.

The small molecules UM171, StemReginin1 (SR1), and valproic acid (VPA), have been previously shown to be capable of expanding long-term (LT) and short-term (ST) engrafting HSPCs, both alone and in combination. Additionally, recent work have suggested that L-ascorbic acid 2-phosphate magnesium salt hydrate (AA2P), UM171, SR1 and VPA can promote the expansion of ST and LT engrafting HSPCs in vitro. These small molecules are functionally divergent and are believed to promote HSPC expansion through different pathways. UM171 is a pyrimidoindole derivative that has been shown to promote proteosomal degradation of the LSD1-CoREST repressor complex, which represses H3K4me2 and H3K27qc marks in hematopoietic stem cells ex vivo. SR1 is an antagonist of the aryl hydrocarbon receptor. AA2P is a long-acting vitamin C (ascorbic acid) derivative that, like ascorbic acid, can enhance the activity of histone deacetylases and DNA hydroxylases. AA2P can also act as a cofactor and enhance the activity of ten-eleven-translocation 2 (TET2) enzyme, which catalyzes the oxidation of cytosine residues that precede DNA demethylation. VPA is a branched chain fatty acid that can inhibit histone deacetylases.

The identification of other agonists of in vitro or ex vivo expansion of HSPCs, particularly those that increase the numbers of LT engraftable cells and cells capable of self-renewal, could revolutionize stem cell graft engineering and gene editing therapeutics.

It would be highly desirable to be provided with compositions as well as methods capable of supporting the in vitro expansion of HSPCs derived from various sources.

### BRIEF SUMMARY

The present disclosure concerns an agonist of a TAM receptor, a ligand of a TAM receptor and/or a compound capable of inducing the expression of a ligand of a TAM receptor for the in vitro or ex vivo expansion of normal HSPCs. The TAM receptor agonist, TAM receptor ligand and/or compound capable of inducing the expression of a TAM receptor ligand can be used alone or in combination with a cytokine and/or a stem cell agonist cocktail.

According to a first aspect, the present disclosure provides an in vitro method of expanding normal HSPCs. The method comprises contacting one or more agonist of a TAM receptor, ligand of a TAM receptor and/or compound capable of inducing the expression of a ligand of a TAM receptor with the normal HSPCs in a culture medium that supports HSPC growth to provide an expanded population of normal HSPCs. In an embodiment, the TAM receptor is a AXL receptor. In an embodiment, the one or more ligand of the AXL receptor comprises a growth arrest 6 (GAS6) polypeptide, a variant of the GAS6 polypeptide having ligand activity towards the AXL receptor or a fragment of the GAS6 polypeptide having ligand activity towards the AXL receptor. In yet another embodiment, the GAS6 polypeptide, the variant of the GAS6 polypeptide having ligand activity toward the AXL receptor or the fragment of the GAS6 polypeptide having ligand activity towards the AXL receptor is present in the culture medium at a concentration of between about 0.1 ng/ml to about 100 ng/mL. In one embodiment, the one or more compound capable of inducing the expression of a ligand of the TAM receptor comprises ascorbic acid or a derivative thereof. In another embodiment, the normal HSPCs comprise human cells. In still another embodiment, the normal HSPCs are derived from cord blood, placenta, bone marrow, peripheral blood, embryonic tissue, induced pluripotent stem cells (IPSCs), or fetal tissue. In still yet another embodiment, the one or more TAM receptor agonist, TAM ligand and/or compound capable of inducing the expression of the TAM receptor ligand, after having contacted the normal HSPCs, is capable of improving the expansion of the normal HSPCs, when compared to control cells. In an embodiment, the in vitro method further comprises contacting the normal HSPCs with a stem cell agonist or a stem cell agonist cocktail comprising StemReginin1, UM171, AA2P and/or VPA. In an embodiment, the normal HSPCs are contacted with the stem cell agonist cocktail prior to contacting the AXL receptor agonist, the AXL receptor ligand or the compound capable of inducing the expression of the AXL receptor ligand. In still another embodiment, the stem cell agonist cocktail (SCAC) comprises: a) about 100 nM to about 5025 nM of StemReginin1; b) about 0.10 nM to about 150 nM of UM171; c) about 0.1 μM to about 2 000 μM of AA2P; and/or d) about 0.01 mM to about 1 mM of valproic acid. In an embodiment, the expanded population of normal HSPCs comprise cells: a) expressing the surface proteins CD34, CD90 and/or CD49f on their cell membrane; b) failing to express the surface protein CD45RA (CD45RA⁻) on their cell membrane; and/or c) expressing the surface protein Endothelial protein C receptor (EPCR) on their cell membrane. In still yet another embodiment, the in vitro method comprises culturing the normal HSPCs in the presence of feeder cells. In still yet another embodiment, the normal HSPCs are cultured: a) in medium supplemented with one or more cytokine; b) in the presence of the stem cell agonist cocktail for at least 2 days; and/or c) in the presence of the GAS6 polypeptide, the variant of the GAS6 polypeptide or the fragment of the GAS6 polypeptide for at least 2 days.

In a second aspect, the present disclosure provides an expanded population of HSPCs obtainable or obtained by the method described herein.

In a third aspect, the present disclosure provides a method for treating a condition in a subject in need thereof comprising: a) providing the expanded population of normal HSPCs provided in the second aspect; and b) grafting the expanded population of normal HSPCs to the subject to treat the condition. In one embodiment, the method further comprises obtaining the normal HSPCs used to provide the expanded population of normal HSPCs from the subject. In another embodiment, the condition being treated by the method comprises: a) a cancer, b) a neural disorder, c) an immune deficiency, d) an auto-immune disorder, e) a metabolic disorder, or f) a genetic disorder. In another embodiment, the method is used to treat a subject that is a human. In another embodiment, the method is used to treat a subject that is an adult. In yet another embodiment, the method is used to treat a subject that is a child.

In a fourth aspect, the present disclosure provides the use of an expanded population of normal HSPCs described herein or prepared by the in vitro method described herein for the manufacture of a medicament for treating a condition in a subject in need thereof. In one embodiment, the normal HSPCs used to provide the expanded population of normal HSPCs are from the subject. In one embodiment, the medicament is for the treatment of: a) a cancer, b) a neural disorder, c) an immune deficiency, d) an auto-immune disorder, e) a metabolic disorder, or f) a genetic disorder. In another embodiment, the subject is a human. In another embodiment, the subject is an adult. In yet another embodiment, the subject that is a child.

In a fifth aspect, the present disclosure provides the use of an expanded population of normal HSPCs described herein or prepared by the in vitro method described herein for treating of a condition in a subject in need thereof. In one embodiment, the normal HSPCs used to provide the expanded population of normal HSPCs are from the subject. In another embodiment, the condition being treated comprises: a) a cancer, b) a neural disorder, c) an immune deficiency, d) an auto-immune disorder, e) a metabolic disorder, or f) a genetic disorder. In another embodiment, the subject is a human. In another embodiment, the method is used to treat a subject that is an adult. In yet another embodiment, the subject is a child.

In a sixth aspect, the present disclosure provides an expanded population of HSPCs described herein or prepared by the in vitro method described herein for treating of a condition in a subject in need thereof. In one embodiment, the condition being treated comprises: a) a cancer, b) a neural disorder, c) an immune deficiency, d) an auto-immune disorder, e) a metabolic disorder, or f) a genetic disorder. In another embodiment, the subject is a human. In another embodiment, the subject is an adult. In yet another embodiment, the subject is a child.

In a seventh aspect, the present disclosure provides a kit for the expansion of normal HSPCs in a culture medium. The kit comprises one or more agonist of a TAM receptor, ligand of a TAM receptor and/or compound capable of inducing the expression of a TAM receptor ligand, and at least one of StemReginin1, UM171, AA2P and valproic acid. In an embodiment, the TAM receptor is a AXL receptor. In one embodiment, the one or more ligand of the AXL receptor comprises a GAS6 polypeptide, a variant of the GAS6 polypeptide having ligand activity towards the AXL receptor or a fragment of the GAS6 polypeptide having ligand activity toward the AXL receptor. In another embodiment, the kit comprises instructions for obtaining an expanded population of normal HSPCs in a culture medium as defined in the method described herein. In another embodiment, the kit further comprises a culture medium, albumin, a buffer, a vitamin, an amino acid, a cytokine, a mineral or trace element, serum and/or a lipid. In yet another embodiment, the kit further comprises an antibiotic, an antifungal and/or a lipoprotein. In one embodiment, the kit comprises the following components that, when added to the normal HSPCs in a culture medium, have the following concentrations: about 0.1 ng/ml to about 25 ng/ml of growth arrest 6 (GAS6) polypeptide, a variant of the GAS6 polypeptide having ligand activity towards the AXL receptor or a fragment of the GAS6 polypeptide having ligand activity towards the AXL receptor; and at least one of about 1 μM to about 10 μM of StemReginin 1; about 0.10nM to about 150 nM of UM171; about 0.1 μM to about 2 000 μM of AA2P; and/or about 0.01 mM to about 1 mM of valproic acid. In another embodiment, the kit comprises components that are packaged individually. In another embodiment, the kit components are packaged together. In yet another embodiment, the kit further comprises feeders cells on which the normal HSPCs can be cultured. In a still further embodiment, the HSPCs produced express elevated levels of DNA repair genes that may increase the efficiency of gene editing within HSPC.

### BRIEF DESCRIPTION OF THE DRAWINGS

Having thus generally described the nature of the invention, reference will now be made to the accompanying drawings, showing by way of illustration, a preferred embodiment thereof, and in which:

FIG. 1A illustrates a representative flow cytometry analysis of UCB hematopoietic stem and progenitor cell (HSPC) cultures supplemented with the different SCACs (see Table 1) at day 14 of culture. CD34+, CD34+CD45RA− and CD34+CD45RA− EPCRHigh (EPCRHigh) subsets.

FIG. 1B illustrates the expansion of total nucleated cells (TNC) in UCB SCAC-supplemented cultures after 14 days of culture. *p<0.05, **p<0.01, ***p<0.001 and ****p<0.0001 by two-way ANOVA.

FIG. 1C illustrates the frequencies (left panel) and net expansion (right panel) of expanded UCB CD34+ HSPC cells after 14 days of culture. *p<0.05, **p<0.01, ***p<0.001 and ****p<0.0001 by two-way ANOVA.

FIG. 1D illustrates the frequencies (left panel) and net expansion (right panel) of expanded UCB CD34+CD45RA− HSPC cells after 14 days of culture. *p<0.05, **p<0.01, ***p<0.001 and ****p<0.0001 by two-way ANOVA.

FIG. 1E illustrates the frequencies (left panel) and net expansion (right panel) of expanded UCB EPCRHigh HSPC cells after 14 days of culture. *p<0.05, **p<0.01, ***p<0.001 and ****p<0.0001 by two-way ANOVA.

FIG. 2A illustrates the expansion of UCB TNC after 14 days of culture. Mean±SEM, one-way ANOVA with Turkey multiple comparisons test *p<0.05, **p<0.01, ***p<0.001 and ****p<0.0001.

FIG. 2B illustrates the expansion of UCB CD34+ cells after 14 days of culture. Mean±SEM, one-way ANOVA with Turkey multiple comparisons test *p<0.05, **p<0.01, ***p<0.001 and ****p<0.0001.

FIG. 2C illustrates the expansion of UCB CD34+CD45RA− cells after 14 days of culture. Mean±SEM, one-way ANOVA with Turkey multiple comparisons test *p<0.05, **p<0.01, ***p<0.001 and ****p<0.0001.

FIG. 2D illustrates the expansion of UCB CD34+CD45RA− EPCRHigh (EPCRHigh) cells after 14 days of culture. Mean±SEM.

FIG. 3A shows the human chimerism measured in the platelets and in the leucocytes present in the peripheral blood of mice at week-3 post-transplantation. Mice were transplanted with the progeny of UCB CD34+ cells expanded for 14 days with indicated SCAC. Representative flow cytometry platelet and leucocyte analyses in nod scid gamma (NSG) mice. Summary of data presented in histograms on the right. Ordinary or Kruskal-Wallis One-way ANOVA with Turkey multiple comparisons test *p<0.05, **p<0.01, ***p<0.001 and ****p<0.0001.

FIG. 3B shows the human chimerism measured in the platelets and in the leucocytes presented in the peripheral blood of mice at week-16 post-transplantation. Platelets and CD45+ leucocytes analyses presented on the left. CD19+ B-cell and CD33+ myeloid leucocytes analyses presented on the right. Ordinary or Kruskal-Wallis One-way ANOVA with Turkey multiple comparisons test *p<0.05, **p<0.01, ***p<0.001 and ****p<0.0001.

FIG. 3C shows the serial multilineage lymphomyeloid human engraftment analysis of bone marrow (BM) cells in serial NSG transplants in primary mice were analysed 18-weeks after transplantation. Each symbol correspond to an individual mouse and the horizontal line presents median, 12-18 mice total per condition. Ordinary or Kruskal-Wallis One-way ANOVA with Turkey multiple comparisons test *p<0.05, **p<0.01, ***p<0.001 and ****p<0.0001.

FIG. 3D shows the multilineage lymphomyeloid engraftment analysis of human BM cells in serial NSG transplants in secondary mice were analysed 12-weeks after transplantation. BM from individual primary mouse transplant were transplanted into other NSG mice. Each symbol correspond to an individual mouse and the horizontal line presents median, 12-18 mice total per condition. Ordinary or Kruskal-Wallis One-way ANOVA with Turkey multiple comparisons test *p<0.05, **p<0.01, ***p<0.001 and ****p<0.0001.

FIG. 3E shows the number of scid repopulating cells (SRC) per 1 000 starting CD34+ cells presented for fresh CB CD34+ cells (NC), or cultures with X2A or C6. *p<0.05, **p<0.01 and ****p<0.0001, One-way ANOVA with Turkey multiple comparisons test. Frequencies of SRC were determined following limit dilution transplant experiments.

FIG. 4A shows the 5-methylcytosine (5MC) levels in CD34+CD45RA− HSPCs obtained from control (STFL) or cultures with AA2P measured via intra-cellular flow cytometry. Median fluorescent intensity (MFI) of 5MC levels in control (STFL) or AA2P cultures are presented. *p<0.05, **p<0.01, ***p<0.001, ****p<0.0001 paired ANOVA test.

FIG. 4B shows the 5-methylcytosine (5MC) levels in CD34+CD45RA− HSPCs obtained from cultures with X2A, X2B or C6 measured via intra-cellular flow cytometry. MFI of 5MC levels within CD34+CD45RA− HSPCs with X2A, X2B or C6 are presented. *p<0.05, **p<0.01, ***p<0.001, ****p<0.0001 paired ANOVA test.

FIG. 4C shows the 5-methylcytosine (5MC) levels in CD34+CD45RA− HSPCs from control (STFL) or cultures with AA2P±Bobcat339 at 30 μM (Bob 30) measured via intra-cellular flow cytometry. MFI of 5MC levels with STFL or AA2P are presented. *p<0.05, **p<0.01, ***p<0.001, ****p<0.0001 paired ANOVA test.

FIG. 4D shows the 5-methylcytosine (5MC) levels in CD34+CD45RA− HSPCs from X2A +Bobcat339 (Bob 30, at 30 μM) or X2B cultures measured via intra-cellular flowcytometry. MFI of 5MC levels within HSPCs with X2A±Bobcat339 or X2B are presented. *p<0.05, **p<0.01, ***p<0.001, ****p<0.0001 paired ANOVA test.

FIG. 4E shows the percentage of methylated (black bars) and unmethylated (white bars) CpG islands (CpG1, CpG2, CpG3 and CpG4) in the GAS6 or the AXL gene of HPSCs cultured in the presence of different SCACs. Representative data from one of the biological replicates is presented.

FIG. 4F shows the expression levels (provided as the relative gene expression, normalized to the STFL control condition by qPCR) of the AXL, GAS6 and PROS1 genes in CD34+CD45RA− HSPCs cultured with C6, X2A, X2B or AA2P. *p<0.05, **p<0.01, ***p<0.001, ****p<0.0001 paired ANOVA test.

FIG. 4G shows the expansion of CD34+CD45RA− HSPCs cultured with STFL, X2A, X2B or C6 in the presence of DMSO or Bobcat339 at 30 μM (Bob 30) from day-10 to day-14. *p<0.05, **p<0.01, ***p<0.001, ****p<0.0001 paired ANOVA test.

FIG. 4H shows the expression levels (provided as the relative gene expression, normalized to the STFL control condition) of the AXL and GAS6 genes in CD34+CD45RA− HSPCs cultured with C6, X2A, X2B or C6 in the presence of DMSO or Bobcat339 at 30 μM (Bob 30) from day-10to day-14. *p<0.05, **p<0.01, ***p<0.001, ****p<0.0001 paired ANOVA test.

FIG. 5A illustrates the proportion of AXL positive cells, as measured by intracellular flow cytometry, within the expanded CD34+CD45RA− HSPCs produced with X2A, X2B or C6. *p<0.05, **p<0.01, ***p<0.001, ****p<0.0001 paired ANOVA test.

FIG. 5B illustrates the proportion of CD34+CD45RA− cells that are positive for activated AXL (pAXL), as measured by intracellular flow cytometry, within the expanded CD34+CD45RA− HSPCs produced with X2A, X2B or C6. *p<0.05, **p<0.01, ***p<0.001, ****p<0.0001 paired ANOVA test.

FIG. 5C illustrates that the expansion of CD34+CD45RA− HSPCs cultured with X2A (left panel) or C6 (right panel) is decreased with increasing concentration of Bemcentinib (indicated in the X axis). Results are shown as expansion normalized to the control culture supplemented with DMSO (vehicle only). *p<0.05, **p<0.01, ***p<0.001, ****p<0.0001 paired ANOVA test.

FIG. 5D shows the expansion of CD34+CD45RA− HSPCs cultured with X2A, X2B or STFL as well as increasing concentration of GAS6 (indicated in the figure legend). GAS6 was added at day 10.

FIG. 5E illustrates that the GAS6 secretion, as measured by Western blot, was increased in cells cultured with X2A when compared to cells cultured with X2B or C6. Representative Western blot analysis of concentrated conditioned media is presented.

FIG. 5F shows the frequency of population, fold expression and net cell counts of HSPCs (CD34+, CD34+CD45RA− and CD34+CD45RA− EPCRHigh) cultured with SM6 in the presence or absence of GAS6 (10 ng/ml added by day 7 of culture).

FIG. 5G illustrates the expansion of total nucleated cells (TNC), CD34+ cells and CD34+CD45RA− cells with X2A, X2B, C6 or SM6 in the presence or not of GAS6 (10 ng/mL added to the culture on day 10) or a GAS6 neutralizing antibody (GAS6 ab). *p<0.05, **p<0.01, ***p<0.001, ****p<0.0001 paired ANOVA test.

FIG. 6A shows that the addition of GAS6 (10 ng/ml) from days 7 to 14 in control STFL cultures improved the expansion of HSPC subsets. *p<0.05 and ****p<0.0001, paired t-test.

FIG. 6B shows that the addition of GAS6 (10 ng/ml) from days 7 to 14 in SR1-containing cultures improved the expansion of TNC and HSPC subsets. *p<0.05 paired t-test.

FIG. 6C shows that the addition of GAS6 (10 ng/ml) from days 7 to 14 in UM171-containing cultures improved the expansion of TNC and HSPC subsets. *p<0.05, **p<0.01 and ****p<0.0001 paired t-test.

FIG. 6D shows that the addition of GAS6 (10 ng/ml) from days 7 to 14 in AA2P-containing cultures improved the expansion of TNC and HSPC subsets. *p<0.05 and **p<0.01 paired t-test.

FIG. 6E shows that the addition of GAS6 (10 ng/mL) from days 7 to 14 in valproic acid (VPA)-containing cultures improved the expansion of TNC and HSPC subsets. *p<0.05 paired t-test.

FIG. 6F shows that the addition of GAS6 (10 ng/ml) from days 7 to 14 in C6-containing cultures improved the expansion of TNC and HSPC subsets. *p<0.05 and ***p<0.001 paired t-test.

FIG. 6G shows that the addition of GAS6 (10 ng/ml) from days 7 to 14 in SM6-containing cultures improved the expansion of TNC and HSPC subsets. *p<0.05, **p<0.01 and ***p<0.001 paired t-test.

FIG. 6H shows that the addition of GAS6 (10 ng/ml) from days 7 to 14 in X2A-containing cultures improved the expansion of TNC and HSPC subsets. *p<0.05 paired t-test.

FIG. 7A illustrates the expansion of TNC, CD34+, CD34+CD45RA− and EPCRHigh HSPC subsets in STFL cultures supplemented with GAS6 at different time point. **p<0.01 ANOVA. GAS6 was added to culture either at day 0 (d0), 4 (d4), 7 (d7) or 10 (d10) and until the end of the culture. Expansion measured at day 14 of culture.

FIG. 7B illustrates the expansion of TNC, CD34+, CD34+CD45RA− and EPCR^{High} HSPC subsets in X2A cultures supplemented with GAS6 at indicated time point. *p<0.05 ANOVA. GAS6 was added to culture either at day 0, 4, 7 or 10 and until the end of the culture. Expansion measured at day 14 of culture.

FIG. 8A illustrates the levels of human platelets measured in the peripheral blood of mice at week-3 and week-16 post-transplantation. Each symbol correspond to an individual mouse and the histogram line presents mean, 9-10 mice total per condition. Ordinary or Kruskal-Wallis One-way ANOVA with Turkey multiple comparisons test **p<0.01.

FIG. 8B illustrates the proportion of human leucocytes (% CD45+) in the peripheral blood of mice at week-3 and week-16 post-transplantation. Ordinary or Kruskal-Wallis One-way ANOVA with Turkey multiple comparisons test *p<0.05, **p<0.01 and ****p<0.0001.

FIG. 8C illustrates the engraftment analysis of human BM cells in primary NSG mice analysed 18-weeks after transplantation. The frequency of human CD45+BM cells are presented. Ordinary or Kruskal-Wallis One-way ANOVA with Turkey multiple comparisons test ****p<0.0001.

FIG. 8D illustrates the multilineage BM lymphomyeloid engraftment analysis. The distribution of the human BM cells (within human CD45+cells) for indicated lineages is presented. Ordinary or Kruskal-Wallis One-way ANOVA with Turkey multiple comparisons test *p<0.05, **p<0.01 and ****p<0.0001.

FIG. 8E illustrates the net number of human CD34+ BM cells measured in primary mice groups. Ordinary or Kruskal-Wallis One-way ANOVA with Turkey multiple comparisons test ****p<0.0001.

FIG. 8F illustrates the net number of human BM colony forming cells (CFU) measured in primary mice groups. Ordinary or Kruskal-Wallis One-way ANOVA with Turkey multiple comparisons test ****p<0.0001.

FIG. 8G illustrates the engraftment analysis of human bone marrow (BM) cells in NSG transplants in secondary mice analysed 12-weeks after transplantation. The frequency of human CD45+ BM cells are presented. Ordinary or Kruskal-Wallis One-way ANOVA with Turkey multiple comparisons test **p<0.01 and ****p<0.0001.

FIG. 9A illustrates the expansion at day 7 of mobilized peripheral blood stem cells (mPBSC) TNC and indicated HSPC subsets achieved in STFL cultures supplemented or not with indicated SCACs.

FIG. 9B illustrates the expansion of mPBSC TNC and HSPC subsets at day 14 in indicated cultures supplemented or not with GAS6 (10 ng/ml). GAS6 was added or not in indicated cultures from day 7-14.

FIG. 10 illustrates that UCB HSPC produced in cultures supplemented with the SCAC (e.g. X2A SCAC) express higher levels of at least a dozen enzymes involved in DNA repair and homologous recombination. This suggest that SCAC may enhance gene-editing in HSPC due to the increased expression of genes normally expressed at lower levels in standard serum-free medium as shown in STFL control condition.

### DETAILED DESCRIPTION

The present disclosure relates to compositions capable of supporting the expansion of a population of HSPCs in vitro or ex vivo. The present disclosure also relates to a kit that comprises at least one stem cell agonist cocktail that can support the expansion of a population of HSPCs in vitro.

The present disclosure thus provides an in vitro method of expanding a population of normal HSPCs by contacting them with one or more agonist of a TAM receptor, ligand of a TAM receptor (e.g. GAS6) or compound capable of inducing expression of a TAM receptor ligand (e.g. AA2P) in a culture medium. As used herein, the term “a population of normal hematopoietic stem and progenitor cells (HSPCs)” refers to a plurality of hematopoietic stem cells and/or hematopoietic progenitor cells of any origin (murine cells, human cells, any other mammalian cells). It also includes cells that have been genetically modified and/or engineered. However, this term does not include hematopoietic stem cells and/or hematopoietic progenitor cells that are characterized, whether functionally and/or genetically, as cancer cells (such as leukemic stem cells). Normal hematopoietic stem cells can be differentiated from leukemic stem cells by certain properties, including: 1) the absence of mutations in oncogenes and/or tumour suppressor genes (e.g. DNMT3A, MLL, NPM1) in their genomes; and/or 2) the absence of a pre-defined differentiation bias/lineage bias that leads to reduced numbers of certain types of progenitor cells (e.g. myeloid-bias or lymphoid-bias).

The methods of the present disclosure concern the expansion of normal HSPCs. As used herein, the terms “expansion”, “expanding” and/or “expanded” refer to increasing the size of the cell population. In some embodiments, the one or more TAM receptor agonist, TAM receptor ligand and/or compound capable of inducing the expression of a TAM receptor ligand, after having contacted the normal HSPCs, is capable of improving the expansion the normal HSPCs when compared to control cells. As used herein, “control cells” refers to normal HSPCs in a culture medium that lacks the agonist of a TAM receptor, ligand of a TAM receptor and/or compound capable of inducing the expression of a TAM receptor ligand, but that may include any diluents, carriers or solvents (e.g. DMSO) that were used to dissolve or solubilize the one or more TAM receptor agonist, TAM receptor ligand or compound capable of inducing the expression of a TAM receptor ligand, prior to it contacting the normal HSPCs. The term “control cells” may also refer to HSPCs in a culture medium that that lacks the agonist of a TAM receptor, ligand of a TAM receptor and/or compound capable of inducing the expression of a TAM receptor ligand, but that further comprises other stem cell agonists cocktails (SCAC) described in the prior art (e.g., C6 as described in the Example). The control cells may also correspond to the normal HSPCs before they are contacted with the agonist of the TAM receptor, ligand of the TAM receptor and/or compound capable of inducing the expression of a TAM receptor ligand. A TAM receptor agonist, TAM receptor ligand and/or compound capable of inducing the expression of a TAM receptor ligand, can be said to have improved the expansion of normal HSPCs when, after having contacted the one or more TAM receptor agonist, TAM receptor ligand and/or compound capable of inducing the expression of a TAM receptor ligand, the size of the population of the normal HSPCs increases by at least about 10% compared to the control cells. As known in the art, TAM receptors include, but are not limited to TYRO3, AXL and MERTK. In an embodiment, the HSPCs that are intended to be expanded express one or more TAM receptors on their surface. For example, the HSPCs can express, prior to expansion, a TAM receptor. In another embodiment, the HSPCs can be induced to express one or more TAM receptors. In another example, the HSPCs that are intended of being expanded can be induced to express a TAM receptor.

The term “TAM receptor ligand” (also referred to as a ligand of a TAM receptor) refers to a natural ligand of a TAM receptor, such as protein S, Tubby, Tubby-like protein 1 (TULP-1), and Galectin-3. The term “a compound capable of inducing the expression of a TAM receptor ligand” (also referred to as a compound capable of inducing the expression of a ligand of a TAM receptor) refers to a small molecule or biologic that is capable of inducing the gene and/or protein expression of a TAM receptor ligand. As used herein, the term “TAM receptor agonist” (also referred to as an agonist of a TAM receptor) refers to a mimetic of a natural ligand of a TAM receptor, that binds to the same site as the natural ligand and produces a similar biological effect as the natural ligand when it binds to the TAM receptor, and/or a small molecule or biologic that can bind a TAM receptor, at any site, and cause the full or partial activation of the TAM receptor. Activation of TAM receptors by an agonist or ligand can be determined by any techniques known in the art, including, but not limited to Western blot analysis using an antibody that recognizes phosphorylation of intracellular tyrosine residues on TAM receptors (e.g. Tyr691, Tyr698, Tyr702, Tyr703, Tyr749 and/or Tyr681). Agonists and/or ligands of TAM receptors may also comprise a small molecule or biologic capable of binding to and activating one or more different TAM receptors.

Previous studies have shown that the AXL receptor in mice and humans is activated through the binding of its natural ligand, the growth arrest-specific protein 6 precursor (GAS6), and by the interaction between GAS6 and phosphatidylserine. When activated by its ligand GAS6, the AXL receptor tyrosine kinase undergoes homodimerisation, autophosphorylates and transphosphorylates its intracellular tyrosine residues (e.g., Tyr691, Tyr698, Tyr702 and/or Tyr703). Activation of the AXL receptor regulates a number of cellular pathways, including several that are critical for the development, growth, and spread of tumors. As a result, the AXL receptor has been deemed an attractive candidate for the development of prognostic biomarkers of malignancies and anticancer therapies.

The term “AXL receptor ligand” (also referred to as a ligand of a AXL receptor) refers to a natural ligand of a AXL receptor (e.g. GAS6). The term “a compound capable of inducing the expression of a AXL receptor ligand” (also referred to as a compound capable of inducing the expression of a ligand of a AXL receptor) refers to a small molecule or biologic that is capable of inducing the gene and/or protein expression of a AXL receptor ligand (e.g. AA2P). As used herein, the term “AXL receptor agonist” (also referred to as an agonist of a AXL receptor) refers to a mimetic of a natural ligand of a AXL receptor, that binds to the same site as the natural ligand and produces a similar biological effect as the natural ligand when it binds to the AXL receptor, and/or a small molecule or biologic that can bind a AXL receptor, at any site, and cause the full or partial activation of the AXL receptor. Activation of AXL receptors by an agonist or ligand can be determined by any techniques known in the art, including, but not limited to Western blot analysis using an antibody that recognizes phosphorylation of intracellular tyrosine residues on AXL receptors (e.g. Tyr691, Tyr698, Tyr702 and/or Tyr703). Agonists and ligands of a AXL receptor may also comprise a small molecule or biologic capable of binding to or activating one or more of the other members of the TAM receptor kinase family members.

In the methods described herein, the TAM receptor agonist, the TAM receptor ligand and/or the compound capable of inducing the expression of a TAM receptor ligand is contacted with the normal HSPCs. The term “contacting” as used herein refers to putting the agonist, whether in a liquid or solid form, into physical contact with the normal HSPCs in culture medium and comprises, for example, adding a soluble form of the agonist directly into the HSPC's culture medium or co-culturing the HSPCs with feeder cells that express and secrete the agonist into the culture medium. In an embodiment, the normal HSPCs are capable of expressing the TAM receptor on their cell surface and the expression of the TAM receptor may have been induced prior to the contact with the TAM receptor agonist, the TAM receptor ligand and/or the compound capable of inducing the expression of the TAM receptor ligand.

In some embodiments, the AXL receptor ligand comprises a GAS6 polypeptide, a variant of a GAS6 polypeptide having ligand activity towards the AXL receptor or a fragment of a GAS6 polypeptide having ligand activity towards the AXL receptor. As used herein, the term a “GAS6 polypeptide” refers to a full-length GAS6 protein from any organism comprising a naturally-occurring amino acid sequence (including amino acid sequences from naturally-occurring protein isoforms) from any organism. In some embodiments, the GAS6 polypeptide is derived from the human gene encoding the growth arrest-specific protein 6 precursor (locus NP 000811.1). In other embodiments, the GAS6 polypeptide may be derived from an ortholog of the human gene encoding the growth arrest-specific protein 6 precursor. A “gene ortholog” is understood to be a gene in a different species that evolved from a common ancestral gene by speciation. In some further embodiments, the GAS6 polypeptide is derived from a paralog of the human gene encoding the GAS6 precursor. A “gene paralog” is understood to be a gene related by duplication within the genome. In the context of the present disclosure, a GAS6 polypeptide would include a polypeptide expressed from a gene ortholog or paralog of the human GAS6 gene and that, when expressed, exhibits the same biological activity as the native human GAS6 polypeptide and, in particular, is capable of activating the AXL receptor.

In an embodiment, a variant of a GAS6 polypeptide can be used as the AXL receptor ligand. The term “a variant of a GAS6 polypeptide” refers to a full-length GAS6 protein, from any organism, that comprises an amino acid sequence that comprises at least one amino acid difference when compared to naturally occurring GAS6 proteins from said organism. The variant of a GAS6 polypeptide described herein may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide for purification of the polypeptide. A variant of the GAS6 polypeptide has at least about 50%, 55%, 60%, 65%, 70%, 75%, 80%, 85%. 90%, 95%, 96%, 97%, 98% or 99% identity to a naturally occurring GAS6 polypeptide and exhibits similar biological activity, such as being capable of activating the AXL receptor. The term “percent (%) identity”, as known in the art, is a relationship between two or more polypeptide sequences or two or more polynucleotide sequences, as determined by comparing the sequences. The level of identity can be determined conventionally using known computer programs. Identity can be readily calculated by known methods, including but not limited to those described in: Computational Molecular Biology (Lesk, A. M., ed.) Oxford University Press, NY (1988); Biocomputing: Informatics and Genome Projects (Smith, D. W., ed.) Academic Press, NY (1993); Computer Analysis of Sequence Data, Part I (Griffin, A. M., and Griffin, H. G., eds.) Humana Press, NJ (1994); Sequence Analysis in Molecular Biology (von Heinje, G., ed.) Academic Press (1987); and Sequence Analysis Primer (Gribskov, M. and Devereux, J., eds.) Stockton Press, NY (1991).

In another embodiment, a fragment of a GAS6 polypeptide can be used as the AXL receptor ligand. The term “a fragment of a GAS6 polypeptide” refers to a polypeptide derived from, but shorter in length than, a full-length GAS6 polypeptide or a full-length variant of a GAS6 polypeptide. A fragment of a GAS6 polypeptide or of a full-length variant of a GAS6 polypeptide has at least about 100, 200, 300, 400, 500 or more consecutive amino acids of the GAS6 polypeptide or the variant of a GAS6 polypeptide. A fragment of a GAS6 polypetide comprises at least one less amino acid residue when compared to the amino acid sequence of the full-length GAS6 polypeptide or variant of a GAS6 polypeptide and still possess the biological activity of the full-length GAS6 polypeptide, such as being capable of activating the AXL receptor. In some embodiments, the “fragments” have at least 50%, 55%, 60%, 65%, 70%, 75%, 80%, 85%, 90%, 95%, 96%, 97%, 98% or 99% identity to the heterologous polypeptides described herein. In some embodiments, fragments of the polypeptides can be employed for producing the corresponding full-length polypeptide by peptide synthesis. Therefore, the fragment of a GAS6 polypeptide can be employed as an intermediate for producing a full-length GAS6 polypeptide. The GAS6 polypeptide, the variant of the GAS6 polypeptide or the fragment of the GAS6 polypeptide may be produced recombinantly.

In a further embodiment, the GAS6 polypeptide, the variant of the GAS6 polypeptide or the fragment of the GAS6 polypeptide is present in the culture medium at a concentration of at least about 0.01, 0.02, 0.03. 0.04, 0.05, 0.06, 0.07, 0.08, 0.08, 0.09, 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100 ng/ml or more. In a further embodiment, the GAS6 polypeptide, the variant of the GAS6 polypeptide or the fragment of the GAS6 polypeptide is present in the culture medium at a concentration of no more than about 100, 95, 90, 85, 80, 75, 70, 65, 60, 55, 50, 45, 40, 35, 30, 25, 20, 15, 10, 9, 8, 7, 6, 5, 4, 3, 2, 1, 0.9, 0.8, 0.7, 0.6, 0.5, 0.4, 0.3, 0.2, 0.1, 0.09, 0.08, 0.07, 0.06, 0.05, 0.04, 0.03, 0.02, 0.01 ng/ml or less. In a further embodiment, the GAS6 polypeptide, the variant of the GAS6 polypeptide or the fragment of the GAS6 polypeptide is present in the culture medium at a concentration of between about 0.01, 0.02, 0.03. 0.04, 0.05, 0.06, 0.07, 0.08, 0.08, 0.09, 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95 ng/ml and about 100, 95, 90, 85, 80, 75, 70, 65,60, 55, 50, 45, 40, 35, 30, 25, 20, 15, 10, 9, 8, 7, 6, 5, 4, 3, 2, 1, 0.9, 0.8, 0.7, 0.6, 0.5, 0.4, 0.3, 0.2, 0.1, 0.09, 0.08, 0.07, 0.06, 0.05, 0.04, 0.03, 0.02, 0.01 ng/ml. In a further embodiment, the GAS6 polypeptide, the variant of the GAS6 polypeptide or the fragment of the GAS6 polypeptide is present in the culture medium at a concentration of between about 0.01 ng/ml to about 100 ng/ml. In one embodiment, the GAS6 polypeptide, the variant of the GAS6 polypeptide or the fragment of the GAS6 polypeptide is present in the culture medium at a concentration of 0.1 ng/ml. In another embodiment, the GAS6 polypeptide, the variant of the GAS6 polypeptide or the fragment of the GAS6 polypeptide is present in the culture medium at a concentration of 2.5 ng/ml. In yet another embodiment, the GAS6 polypeptide, the variant of the GAS6 polypeptide or the fragment of the GAS6 polypeptide is present in the culture medium at a concentration of 10 ng/ml. In still another embodiment, the GAS6 polypeptide, the variant of the GAS6 polypeptide or the fragment of the GAS6 polypeptide is present in the culture medium at a concentration of 25 ng/ml.

The method of the present disclosure also concerns methods in which a compound capable of inducing the expression of the ligand of the TAM receptor is contacted with the normal HSPCs to favor their expansion. As used in the context of the present disclosure, “a compound capable of inducing the expression of the ligand of the TAM receptor” refers to a compound or a biological molecule capable of increase the expression of the gene encoding the ligand of the TAM receptor. In some embodiments, the compound or biological molecule “capable of inducing the expression of the ligand of the TAM receptor” is also capable of inducing the expression of the TAM receptor recognizing such ligand. The compound or the biological molecule can induce the expression of the ligand of the TAM receptor in a specific or in a non-specific manner. In embodiments in which the TAM receptor is the AXL receptor, one compound that can be used to induce the expression of the ligand of the AXL receptor (e.g., GAS6) can be ascorbic acid or a derivative of ascorbic acid. In an embodiment, when ascorbic acid or a derivative of ascorbic acid is used to induce the expression of the ligand of the AXL receptor, it is not used in combination with other components of a SACS cocktail. However, in some alternative embodiments, when ascorbic acid or a derivative of ascorbic acid is used to induce the expression of the ligand of the AXL receptor, it can be used in combination with other components of a SCAC cocktail. Ascorbic acid derivatives include, but are not limited to, AA2P, ascorbic acid, sodium ascorbyl phosphate, ascorbyl palmitate, retinyl ascorbate, tetrahexyldecyl ascorbate, and magnesium ascorbyl phosphate. In yet another embodiment, the compound capable of inducing the expression of the ligand of the TAM receptor is used at a concentration of at least about 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 600, 700, 800, 900, 1000, 1100, 1200, 1300, 1400, 1500, 1600, 1700, 1800, 1900, 2000 μM or more. In a further embodiment, the compound capable of inducing the expression of the ligand of the TAM receptor is used at a concentration of no more than about 2000, 1900, 1800, 1700, 1600, 1500, 1400, 1300, 1200, 1100, 1000, 900, 800, 700, 600, 500, 400, 300, 200, 100, 95, 90, 85, 80, 75, 70, 65, 60, 55, 50, 45, 40, 35, 30, 25, 20, 15, 10, 9, 8, 7, 6, 5, 4, 3, 2, 1, 0.9, 0.8, 0.7, 0.6, 0.5, 0.4, 0.3, 0.2, 0.1 μM or less. In a further embodiment, the compound capable of inducing the expression of the ligand of the TAM receptor is used at a concentration of between about 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 600, 700, 800, 900, 1000, 1100, 1200, 1300, 1400, 1500, 1600, 1700, 1800, 1900, 2000 μM and about 2000, 1900, 1800, 1700, 1600, 1500, 1400, 1300, 1200, 1100, 1000, 900, 800, 700, 600, 500, 400, 300, 200, 100, 95, 90, 85, 80, 75, 70, 65, 60, 55, 50, 45, 40, 35, 30, 25, 20, 15, 10, 9, 8, 7, 6, 5, 4, 3, 2, 1, 0.9, 0.8, 0.7, 0.6, 0.5, 0.4, 0.3, 0.2, 0.1 μM.

In some embodiments, the normal HSPCs are of human origin. In some embodiments, the normal HSPCs are derived from umbilical cord blood, placenta, bone marrow, peripheral blood, embryonic tissue, induced pluripotent stem cells (iPSCs), or fetal tissue. As used herein, “derived from” means having been received from, having been obtained from or having arisen from a particular source. In some embodiments, the normal HSPCs can be obtained from umbilical cord blood, placenta, bone marrow, peripheral blood (such as mobilized stem cells), embryonic tissue, iPSCs or fetal tissue prior to contacting them with one or more agonist of a TAM receptor, a ligand of a TAM receptor and/or a compound capable of inducing the expression of a TAM receptor ligand in a culture medium in order to obtain an expanded population of normal HSPCs. In some embodiments, the normal HSPCs are pre-enriched, in order to remove mature cells, using conventional methods that are known in the art and that provide 25-95% purity.

The combination of UM171, StemReginin1 (SR1), L-ascorbic acid 2-phosphate magnesium salt hydrate (AA2P) and valproic acid (VPA) is capable of promoting HSPC expansion in vitro or ex vivo, albeit through different pathways. In some embodiments, in addition to the one or more TAM receptor agonist, TAM receptor ligand and/or compound capable of inducing the expression of a TAM receptor, the normal HSPCs are contacted with a stem cell agonist cocktail (SCAC) comprising UM171, SR1, AA2P and/or VPA. In some further embodiments, the normal HSPCs contact the SCAC prior to contacting the one or more TAM receptor agonist, TAM receptor ligand and/or compound capable of inducing the expression of a TAM receptor. In a further embodiment, the SCAC comprises a concentration of SR1 of at least about 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100 nM or more. In a further embodiment, the SCAC comprises a concentration of SR1 of no more than 5100, 5000, 4900, 4800, 4700, 4600, 4500, 4400, 4300, 4200, 4100, 4000, 3900, 3800, 3700, 3600, 3500, 3400, 3300, 3200, 3100, 3000, 2900, 2800, 2700, 2600, 2500, 2400, 2300, 2200, 2100, 2000, 1900, 1800, 1700, 1600, 1500, 1400, 1300, 1200, 1100, 1000, 900, 800, 700, 600, 500, 400, 300, 200, 100 nM or less. In a further embodiment, the SCAC comprises a concentration of SR1 of between about 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100 nM and about 5100, 5000, 4900, 4800, 4700, 4600, 4500, 4400, 4300, 4200, 4100, 4000, 3900, 3800, 3700, 3600, 3500, 3400, 3300, 3200, 3100, 3000, 2900, 2800, 2700, 2600, 2500, 2400, 2300, 2200, 2100, 2000, 1900, 1800, 1700, 1600, 1500, 1400, 1300, 1200, 1100, 1000, 900, 800, 700, 600, 500, 400, 300, 200, 100 nM.

In a further embodiment, the SCAC comprises a concentration of UM171 of at least about 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150 nM or more. In a further embodiment, the SCAC comprises a concentration of UM171 of no more than about 150, 145, 140, 135, 130, 125, 120, 115, 110, 105, 100, 95, 90, 85, 80, 75, 70, 65, 60, 55, 50, 45, 40, 35, 30, 25, 20, 15, 10, 9, 8, 7, 6, 5, 4, 3, 2, 1, 0.9, 0.8, 0.7, 0.6, 0.5, 0.4, 0.3, 0.2, 0.1 nM or less. In a further embodiment, the SCAC comprises a concentration of UM171 of between about 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150 nM and about 150, 145, 140, 135, 130, 125, 120, 115, 110, 105, 100, 95, 90, 85, 80, 75, 70, 65, 60, 55, 50, 45, 40, 35, 30, 25, 20, 15, 10, 9, 8, 7, 6, 5, 4, 3, 2, 1, 0.9, 0.8, 0.7, 0.6, 0.5, 0.4, 0.3, 0.2, 0.1 nM.

While the Examples have shown that AA2P could be useful in the cocktail, it is understood that any vitamin C derivatives can be used in the SCAC. Additional vitamin C derivatives include, but are not limited to ascorbic acid, sodium ascorbyl phosphate, ascorbyl palmitate, retinyl ascorbate, tetrahexyldecyl ascorbate, and magnesium ascorbyl phosphate. In a further embodiment, the SCAC comprises a concentration of AA2P of at least about 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 600, 700, 800, 900, 1000, 1100, 1200, 1300, 1400, 1500, 1600, 1700, 1800, 1900, 2000 μUM or more. In a further embodiment, the SCAC comprises a concentration of AA2P of no more than about 2000, 1900, 1800, 1700, 1600, 1500, 1400, 1300, 1200, 1100, 1000, 900, 800, 700, 600, 500, 400, 300, 200, 100, 95, 90, 85, 80, 75, 70, 65, 60, 55, 50, 45, 40, 35, 30, 25, 20, 15, 10, 9, 8, 7, 6, 5, 4, 3, 2, 1, 0.9, 0.8, 0.7, 0.6, 0.5, 0.4, 0.3, 0.2, 0.1 μM or less. In a further embodiment, the SCAC comprises a concentration of AA2P of between about 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 600, 700, 800, 900, 1000, 1100, 1200, 1300, 1400, 1500, 1600, 1700, 1800, 1900, 2000 μM and about 2000, 1900, 1800, 1700, 1600, 1500, 1400, 1300, 1200, 1100, 1000, 900, 800, 700, 600, 500, 400, 300, 200, 100, 95, 90, 85, 80, 75, 70, 65, 60, 55, 50, 45, 40, 35, 30, 25, 20, 15, 10, 9, 8, 7, 6, 5, 4, 3, 2, 1, 0.9, 0.8, 0.7, 0.6, 0.5, 0.4, 0.3, 0.2, 0.1 μM.

The TAM agonist can be used with a cocktail lacking VPA. However, in some embodiments, the TAM agonist can be used with a SCAC comprising VPA. In some further embodiments, the SCAC comprises a concentration of VPA of at least about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1 mM or more. In some further embodiments, the SCAC comprises a concentration of VPA of no more than about 1, 0.9, 0.8, 0.7, 0.6, 0.5, 0.4, 0.3, 0.2, 0.1, 0.09, 0.08, 0.07, 0.06, 0.05, 0.04, 0.03, 0.02, 0.1 mM or less. In some further embodiments, the SCAC comprises a concentration of VPA of between about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1 mM and about 1, 0.9, 0.8, 0.7, 0.6, 0.5, 0.4, 0.3, 0.2, 0.1, 0.09, 0.08, 0.07, 0.06, 0.05, 0.04, 0.03, 0.02, 0.1 mM.

In some embodiments, the SCAC comprises about 100 nM to about 5025 nM of SR1, about 0.10 nM to about 150 nM of UM171, about 0.1 μM to about 2000 μM of AA2 P, and about 0.01 mM to about 1 mM of VPA. In one embodiment, the SCAC comprises about 5023 nM of SR1, about 0.35 nM of UM171, about 0.502 mM of VPA and about 1000 μM of AA2P. In another embodiment, the SCAC comprises about 5023 nM of SR1, about 125.63 nM of UM171, about 0.01 mM of VPA and about 1000 μM of AA2P. In a further embodiment, the SCAC comprises about 1000 nM of SR1, about 38 nM of UM171, about 0.125 mM of VPA and about 250 μM of AA2P. In yet a further embodiment, the SCAC comprises about 2500 nM of SR1, about 62 nM of UM171, about 0.01 mM of VPA and about 1000 μM of AA2P. In still another embodiment, the SCAC comprises about 2500 nM of SR1, about 62 nM of UM171, about 0.01 mM of VPA and about 0.1 μM of AA2P.

The in vitro methods described above provide for an expanded population of normal HSPCs which share similarities with the normal HSPCs from which they are derived. Normal HSPCs can be identified and characterized based on the pattern of markers that they express on their cell surface. For example, normal human HSPCs express the cell surface protein CD34, which has been used as a marker to identify and purify the cells for various purposes including HSCT. CD90 is expressed on the surface of various types of human stem cells found in different tissues. In combination with the expression of CD34, expression of CD90 on the cell surface is a reliable marker of normal HSCs. CD49f is a protein expressed on the surface of HSCs that is associated with increased efficiency of long-term multilineage grafts. The endothelial protein C receptor (EPCR), also known as CD201, is also expressed on the surface of HSC. EPCR has been shown to help guide transplanted HSCs to stem cell niches in the bone marrow and, therefore, high expression of this marker on the cell surface of HSCs has been associated with increased retention following engraftment. The absence of or reduced expression of the cell surface marker CD45RA has been used in order to select normal HSPCs, as opposed to leukemic stem cells. The level of expression of any one of the above-mentioned cell surface markers on the surface of a given HSC or HPC may be determined, for example, by flow cytometry using a fluorescently-labeled antibody that is specific for the marker in question. In some embodiments, the expanded population of normal HSPCs comprises cells that express the surface proteins CD34, CD90 and/or CD49f on their cell membranes. In some embodiments, the expanded population of normal HSPCs comprise cells that express at least two of the surface proteins CD34, CD90 and CD49f. In other embodiments, the expanded population of normal HSPCs comprise cells that express all three of the surface proteins CD34, CD90 and CD49f. In other embodiments, the expanded population of normal HSPCs comprise cells that lack or express low levels of CD45RA. In other embodiments the expanded population of normal HSPCs comprise cells that express high levels of the surface protein EPCR. In yet other embodiments, the expanded population of normal HSPCs comprise cells that express one or more of the surface proteins CD34, CD90 and CD49f and that do not express, or that express low levels, of the surface protein CD45RA on their cells surface. In other embodiments, the expanded population of normal HSPCs comprise cells that express one or more of the surface proteins CD34, CD90 and CD49f and that express high levels of the surface protein EPCR. In some other embodiments, the expanded population of normal HSPCs comprise cells that do not express, or that express low levels, of the surface protein CD45RA on their cells surface and that express high levels of the surface protein EPCR. In yet further embodiments, the expanded population of normal HSPCs comprise cells that express one or more of the surface proteins CD34, CD90 and/or CD49f; do not express, or that express low levels, of the surface protein CD45RA on their cells surface; and express high levels of the surface protein EPCR. The in vitro methods described herein may further comprise determining the level of expression of the surface markers CD34, CD90, CD49f, CD45RA, and EPCR on the cell membranes of the cells that comprise the expanded population of normal HSPCs.

As used herein, the term “feeder cells” refers to non-proliferating cells which produce growth factors, adhesion molecules, extracellular matrix components or other factors that help support the growth and expansion of HSPCs. In some embodiments, the in vitro method of expanding a population of normal HSPCs further comprises culturing the normal HSPCs in the presence of feeder cells. In some embodiments, the feeder cells express GAS6. In other embodiments, the feeder cells do not express GAS6. In yet other embodiments, the normal HSPCs are not cultured in the presence of feeder cells (in a feeder-free method).

In some embodiments, the in vitro method of expanding a population of normal HSPCs comprises culturing the normal HSPCs in culture medium supplemented with one or more cytokines. Examples of suitable cytokines include interleukins (e.g. IL-3, IL-6 and others ILs), stem cell factors (SFO), thrombopoietin (TPO), colony stimulating factors (e.g. GM-CSF, G-CSF, M-CSF), transforming growth factors (TGF-β), flt-3/flk-2 ligand (FL), interferons (e.g. IFN-α, IFN-β, IFN-γ) leukemia inhibitory factor (LIF) and tumour necrosis factors (e.g. TNF-α, TNF-β). The concentration of the one or more cytokines can vary but it is usually in a range between about 0.1 to about 200 ng/ml. The one or more cytokines added in the culture medium should allow for the expansion of HSPCs that are engraftable and/or that maintain self-renewal capabilities. In other embodiments, the in vitro method comprises culturing the normal HSPCs in the presence of the stem cell agonist cocktail (UM171, SR1, AA2P and/or VPA) for at least 2 days. In yet other embodiments, the in vitro method comprises culturing the normal HSPCs in the presence of the GAS6 polypeptide, a variant of the GAS6 polypeptide or a fragment of the GAS6 polypeptide for at least 2 days. In some embodiments, the in vitro method comprises first culturing the normal HSPCs in medium supplemented with one or more cytokine, then culturing them in the presence of the stem cell agonist cocktail for at least 2 days and, lastly, culturing them in the presence of the GAS6 polypeptide, a variant of the GAS6 polypeptide or a fragment of the GAS6 polypeptide for at least 2 days. In other embodiments, the in vitro method comprises culturing the normal HSPCs in the presence of an individual stem cell agonist (e.g. UM171, SR1, AA2P and/or VPA etc.) and in the presence of the GAS6 polypeptide, a variant of the GAS6 polypeptide or a fragment of the GAS6 polypeptide for at least 2 days. In other embodiments, the in vitro method comprises first culturing the normal HSPCs in medium supplemented with one or more cytokine, then culturing them in the presence of the GAS6 polypeptide, a variant of the GAS6 polypeptide or a fragment of the GAS6 polypeptide for at least 2 days and, lastly, culturing them in the presence of the stem cell agonist cocktail for at least 2 days. In some other embodiments, the in vitro method comprises first culturing the normal HSPCs in a medium supplemented with one or more cytokine and then culturing the cells in the presence of both the stem cell agonist cocktail and the GAS6 polypeptide, a variant of the GAS6 polypeptide or a fragment of the GAS6 polypeptide, at the same time, for at least 2 days.

In yet another embodiment, the method is performed is the absence of a serum (e.g., serum free conditions). In still another embodiment, the method is performed at a temperature higher than 33° C. For example, the method can be performed by maintaining the cells at a temperature equal to or above 34, 35, 36, 37, 38, 39 or 40° C. In a specific example, the method can be performed at a temperature equal to about 37° C.

The expanded population of normal HSPCs obtained or obtainable by any of the above-mentioned in vitro methods exhibit unique transcriptional profiles that comprise the differential expression of hundreds of genes compared to non-expanded HSPCs or HSPCs expanded by other methods (Tables 2-5). The expanded population of normal HSPCs obtained or obtainable by any of the above-mentioned in vitro methods have also been shown to have an improved ability to repopulate human leukocytes in peripheral blood over the short-term and to increase bone marrow engraftment activity over the long-term when compared to non-expanded or HSPCs expanded by other methods.

The present disclosure also provides a method for treating a condition in a subject in need thereof that comprises providing the expanded population of normal HSPCs obtained or obtainable by any of the above-mentioned in vitro methods and grafting said population of expanded HSPCs to the subject in order to treat the condition. In some embodiments, the method of treatment further comprises obtaining the normal HSPCs used to provide the expanded population of normal HSPCs from the subject. These normal HSPCs may be obtained from the subject's umbilical cord blood, placenta, bone marrow, peripheral blood, embryonic tissue, induced pluripotent stem cells (iPSCs), or fetal tissue In some embodiments, the method is used for treating a cancer, a neural disorder, an immune deficiency, an autoimmune disorder, a metabolic disorder and or a genetic disorder in the subject in need thereof. Some examples of cancers that may be treated using the expanded population of normal HSPCs obtained or obtainable by the above mentioned in vitro method include cancers such as multiple myeloma, acute myeloid leukemia, chronic myeloid leukemia, acute lymphoblastic leukemia, Hodgkin lymphoma (relapsed, refractory), non-Hodgkin lymphoma (relapsed, refractory), neuroblastoma, Ewing sarcoma, myelodynaplastic syndromes, gliomas, and other solid tumors. For some subjects with cancer, the expanded population of normal HSPCs may be used to rebuild their immune system after they have undergone chemotherapy or chemoradiotherapy. Some examples of non-cancerous conditions that may be treated using the expanded population of normal HSPCs obtained or obtainable by the above mentioned in vitro method include thalassemia, sickle cell anemia, aplastic anemia, Fanconi anemia, malignant infantile osteopetrosis, mucopolysaccharidosis and pyruvate kinase deficiency. In some embodiments, the method for treating a condition in the subject comprises determining the need of the subject to receive an expanded population of HSPCs. In other embodiments, the method for treating a condition in the subject comprises receiving a second dose of an expanded population of normal HSPCs after having received a first dose of the same.

In some embodiments, the subject being treated using the above-mentioned method is a human being. In some embodiments, the subject is an adult. In other embodiments, the subject is a child.

In some embodiments, the kit produce a population of HSPCs programmed to express high levels of DNA repair enzymes that make the HSPCs more amenable to gene editing. Gene editing of HSPC could be used to treat genetic disorders that originate in the hematopoietic system such as but not limited to beta-globin disorders (e.g. sickle cell disease), immune disorders (e.g. XHIM), cancer (e.g. AML, ALL) and infectious diseases (e.g. HIV).

The present disclosure further provides a kit for the expansion of normal HSPCs in a culture medium. The kit comprises one or more agonist of the TAM receptor, one or more ligand of a TAM receptor and/or one or more compound capable of inducing the expression of a TAM receptor ligand, SR1, UM171, AA2P and/or VPA. In some embodiments, the TAM receptor is an AXL receptor. In a further embodiment, the kit comprises a GAS6 polypeptide, a variant of the GAS6 polypeptide or a fragment of the GAS6 polypeptide (to be added to the culture medium at a concentration of at least about 0.01, 0.02, 0.03. 0.04, 0.05, 0.06, 0.07, 0.08, 0.08, 0.09, 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100 ng/ml or more). In a further embodiment, the GAS6 polypeptide, the variant of the GAS6 polypeptide or the fragment of the GAS6 polypeptide is added to the culture medium at a concentration of no more than about 100, 95, 90, 85, 80, 75, 70, 65, 60, 55, 50, 45, 40, 35, 30, 25, 20, 15, 10, 9, 8, 7, 6, 5, 4, 3, 2, 1, 0.9, 0.8, 0.7, 0.6, 0.5, 0.4, 0.3, 0.2, 0.1, 0.09, 0.08, 0.07, 0.06, 0.05, 0.04, 0.03, 0.02, 0.01 ng/mL or less. In a further embodiment, the GAS6 polypeptide, the variant of the GAS6 polypeptide or the fragment of the GAS6 polypeptide is added to the culture medium at a concentration of between about 0.01, 0.02, 0.03. 0.04, 0.05, 0.06, 0.07, 0.08, 0.08, 0.09, 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95 ng/mL and about 100, 95, 90, 85, 80, 75, 70, 65,60, 55, 50, 45, 40, 35, 30, 25, 20, 15, 10, 9, 8, 7, 6, 5, 4, 3, 2, 1, 0.9, 0.8, 0.7, 0.6, 0.5, 0.4, 0.3, 0.2, 0.1, 0.09, 0.08, 0.07, 0.06, 0.05, 0.04, 0.03, 0.02, 0.01 ng/mL. In a further embodiment, the kit comprises a GAS6 polypeptide, a variant of the GAS6 polypeptide or a fragment of the GAS6 polypeptide (to be added to the culture medium at a concentration of between about 0.01 ng/ml to about 100 ng/ml). In one embodiment, the kit comprises a GAS6 polypeptide, a variant of the GAS6 polypeptide or a fragment of the GAS6 polypeptide (to be added to the culture medium at a concentration of 0.1 ng/ml). In one embodiment, the kit comprises a GAS6 polypeptide, a variant of the GAS6 polypeptide or a fragment of the GAS6 polypeptide (to be added to the culture medium at a concentration of 2.5 ng/ml). In one embodiment, the kit comprises a GAS6 polypeptide, a variant of the GAS6 polypeptide or a fragment of the GAS6 polypeptide (to be added to the culture medium at a concentration of 10 ng/ml). In one embodiment, the kit comprises a GAS6 polypeptide, a variant of the GAS6polypeptide or a fragment of the GAS6 polypeptide (to be added to the culture medium at a concentration of 25 ng/ml).

In a further embodiment, the kit comprises SR1 to be added to the culture medium at a concentration of at least about 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100 nM or more. In a further embodiment, the kit comprises SR1 to be added to the culture medium at a concentration of no more than 5100, 5000, 4900, 4800, 4700, 4600, 4500, 4400, 4300, 4200, 4100, 4000, 3900, 3800, 3700, 3600, 3500, 3400, 3300, 3200, 3100, 3000, 2900, 2800, 2700, 2600, 2500, 2400, 2300, 2200, 2100, 2000, 1900, 1800, 1700, 1600, 1500, 1400, 1300, 1200, 1100, 1000, 900, 800, 700, 600, 500, 400, 300, 200, 100 nM or less. In a further embodiment, the kit comprises SR1 to be added to the culture medium at a concentration of between about 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100 nM and about 5100, 5000, 4900, 4800, 4700, 4600, 4500, 4400, 4300, 4200, 4100, 4000, 3900, 3800, 3700, 3600, 3500, 3400, 3300, 3200, 3100, 3000, 2900, 2800, 2700, 2600, 2500, 2400, 2300, 2200, 2100, 2000, 1900, 1800, 1700, 1600, 1500, 1400, 1300, 1200, 1100, 1000, 900, 800, 700, 600, 500, 400, 300, 200, 100 nM.

In a further embodiment, the kit comprises UM171 (to be added to the culture medium at a concentration of at least about 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150 nM or more). In a further embodiment, the kit comprises UM171 (to be added to the culture medium at a concentration of no more than about 150, 145, 140, 135, 130, 125, 120, 115, 110, 105, 100, 95, 90, 85, 80, 75, 70, 65, 60, 55, 50, 45, 40, 35, 30, 25, 20, 15, 10, 9, 8, 7, 6, 5, 4, 3, 2, 1, 0.9, 0.8, 0.7, 0.6, 0.5, 0.4, 0.3, 0.2, 0.1 nM or less). In a further embodiment, the kit comprises UM171 (to be added to the culture medium at a concentration of between about 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150 nM and about 150, 145, 140, 135, 130, 125, 120, 115, 110, 105, 100, 95, 90, 85, 80, 75, 70, 65, 60, 55, 50, 45, 40, 35, 30, 25, 20, 15, 10, 9, 8, 7, 6, 5, 4, 3, 2, 1, 0.9, 0.8, 0.7, 0.6, 0.5, 0.4, 0.3, 0.2, 0.1 nM).

While the Examples have shown that AA2P could be useful in the cocktail, it is understood that any vitamin C derivatives can be used in the SCAC. Additional vitamin C derivatives include, but are not limited to sodium ascorbyl phosphate, ascorbyl palmitate, retinyl ascorbate, tetrahexyldecyl ascorbate, and magnesium ascorbyl phosphate. In a further embodiment, the kit comprises AA2P (to be added to the culture medium at a concentration of at least about 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 600, 700, 800, 900, 1000, 1100, 1200, 1300, 1400, 1500, 1600, 1700, 1800, 1900, 2000 μM or more). In a further embodiment, the kit comprises AA2P (to be added to the culture medium at a concentration of no more than about 2000, 1900, 1800, 1700, 1600, 1500, 1400, 1300, 1200, 1100, 1000, 900, 800, 700, 600, 500, 400, 300, 200, 100, 95, 90, 85, 80, 75, 70, 65, 60, 55, 50, 45, 40, 35, 30, 25, 20, 15, 10, 9, 8, 7, 6, 5, 4, 3, 2, 1, 0.9, 0.8, 0.7, 0.6, 0.5, 0.4, 0.3, 0.2, 0.1 μM or less). In a further embodiment, the kit comprises AA2P (to be added to the culture medium at a concentration of between about 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 600, 700, 800, 900, 1000, 1100, 1200, 1300, 1400, 1500, 1600, 1700, 1800, 1900, 2000 μM and about 2000, 1900, 1800, 1700, 1600, 1500, 1400, 1300, 1200, 1100, 1000, 900, 800, 700, 600, 500, 400, 300, 200, 100, 95, 90, 85, 80, 75, 70, 65, 60, 55, 50, 45, 40, 35, 30, 25, 20, 15, 10, 9, 8, 7, 6, 5, 4, 3, 2, 1, 0.9, 0.8, 0.7, 0.6, 0.5, 0.4, 0.3, 0.2, 0.1 μM). In some further embodiments, the kit comprises VPA (to be added to the culture medium at a concentration of at least about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1 mM or more).

The TAM agonist can be used with a cocktail lacking VPA. However, in some embodiments, the TAM agonist can be used with a SCAC comprising VPA. In some further embodiments, the kit comprises VPA (to be added to the culture medium at a concentration of no more than about 1, 0.9, 0.8, 0.7, 0.6, 0.5, 0.4, 0.3, 0.2, 0.1, 0.09, 0.08, 0.07, 0.06, 0.05, 0.04, 0.03, 0.02, 0.1 mM or less). In some further embodiments, the kit comprises VPA (to be added to the culture medium at a concentration of between about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1 mM and about 1, 0.9, 0.8, 0.7, 0.6, 0.5, 0.4, 0.3, 0.2, 0.1, 0.09, 0.08, 0.07, 0.06, 0.05, 0.04, 0.03, 0.02, 0.1 mM).

In some embodiments, the kit comprises SR1 (to be added to the culture medium at a concentration of about 1 μM to about 10 μM); UM171 (to be added to culture medium at a concentration of about 0.1 nM to about 150 nM); AA2P (to be added to the culture medium at a concentration of about 0.1 μM to about 2 000 M); VPA (to be added to the culture medium at a concentration of 0.01 mM to about 1 mM); and a GAS6 polypeptide, a variant of a GAS6 peptide or a fragment of a GAS6 peptide, (to be added to the culture medium at a concentration of about 0.1 to about 25 ng/ml). In other embodiments, the kit comprises SR1 (to be added to the cell culture medium at a concentration of about 5023 nM), UM171 (to be added to the cell culture medium at a concentration of about 0.35 nM), VPA (to be added to the cell culture medium at a concentration of about 0.502 mM), and AA2P (to be added to the cell culture medium at a concentration of about 1000 μM). In another embodiment, the kit comprises SR1 (to be added to the cell culture medium at a concentration of about 5023 nM), UM171 (to be added to the cell culture medium at a concentration of about 125.63 nM), VPA (to be added to the cell culture medium at a concentration of about 0.01 mM), and AA2P (to be added to the cell culture medium at a concentration of about 1000 μM). In a further embodiment, the kit comprises SR1 (to be added to the cell culture medium at a concentration of about 1000 nM), UM171 (to be added to the cell culture medium at a concentration of about 38 nM), VPA (to be added to the cell culture medium at a concentration of about 0.125 mM), and AA2P (to be added to the cell culture medium at a concentration of about 250 μM). In yet a further embodiment, the kit comprises SR1 (to be added to the cell culture medium at a concentration of about 2500 nM), UM171 (to be added to the cell culture medium at a concentration of about 62 nM), VPA (to be added to the cell culture medium at a concentration of about 0.01 mM), and AA2P (to be added to the cell culture medium at a concentration of about 1000 μM). In still another embodiment, the kit comprises SR1 (to be added to the cell culture medium at a concentration of about 2500 nM), UM171 (to be added to the cell culture medium at a concentration of about 62 nM), VPA (to be added to the cell culture medium at a concentration of about 0.01 mM), and AA2P (to be added to the cell culture medium at a concentration of about 0.1 μM). In some embodiments, the kit further comprises instructions for obtaining an expanded population of normal HSPCs, in a culture medium comprising SR1, UM171, AA2P, VPA and/or a GAS6 polypeptide, a variant of a GAS6 peptide or a fragment of a GAS6 peptide, using any of the in vitro methods described above.

In some embodiments, the kit of the present disclosure comprises a cell culture medium. Examples of suitable cell culture mediums include biological fluids and tissue extracts (e.g. plasma, serum, placental cord serum, amniotic fluid, embryo extracts), balanced salt solutions (e.g. PBS, DPBS, HBSS, EBSS), artificial medium (e.g. StemSpan™ SFEM, MEM, DMEM, Ham's F-12, Medium, RPMI-1640, IMDM, Medium 199) or any combinations thereof. In an embodiment, the cell culture medium is substantially free from serum (e.g., it has not been supplemented with serum). In some embodiments, the kit comprises a serum including human serum, bovine serum, fetal bovine serum, newborn calf serum and horse serum. In some embodiments, the kit comprises albumin which may, for example, be derived from a human or another mammal (e.g., human serum albumin, bovine serum albumin). In some embodiments, the kit comprises a buffer. Examples of buffers that are suitable for use in cell culture include, but are not limited to, phosphate buffers (e.g. PBS), HEPES, MOPS, MES, BES, bicarbonate buffers, bicine buffers and tricine buffers. In some embodiments, the kit may further comprise vitamin, including vitamin A, any and all B group vitamins, vitamin C, vitamin D, vitamin E and/or vitamin K. In some embodiments, the kit comprises an amino acid, an amino acid derivative or a dipeptide of an amino acid including L-arginine, L-cysteine, L-cystine, L-glutamine, L-histidine, L-isoleucine, L-leucine, L-lysine, L-methionine, L-phenylalanine, L-threonine, L-tryptophan, L-tyrosine, L-valine, glycine, L-alanine, L-asparagine, L-aspartic acid, L-glutamic acid, L-proline, L-serine, phosphor-L-tyrosine, s-sulfo-L-cysteine, L-alanyl L-tyrosine and/or L-alanyl L-glutamine. In some embodiments, the kit comprises a cytokine including interleukins (e.g. IL-1 to IL-33), stem cell factors (SFO), thrombopoietin (TPO), colony stimulating factors (e.g. GM-CSF, G-CSF, M-CSF), transforming growth factors (TGF-β), flt-3/flk-2 ligand (FL), and/or interferons (e.g. IFN-α, IFN-β, IFN-γ) leukemia inhibitory factor (LIF) and tumour necrosis factors (e.g. TNF-α, TNF-β). In some embodiments, the kit comprises a mineral or trace element including sodium, potassium, chloride, magnesium, calcium, phosphorus, selenium, zinc, iron, cooper and/or manganese. In some embodiments, the kit comprises a lipid such as arachidonic acid, cholesterol, DL-α-tocopherol acetate, linoleic acid, linolenic acid, myristic acid, oleic acid, palmitoleic acid, palmitic acid and/or stearic acid.

In yet other embodiments, the kit further comprises an antibiotic such as ampicilin, erythromycin, gentamycin, kanamycin, neomycin, nystatin, penicillin, streptomycin, polymyxin B, and/or tetracycline. In other embodiments, the kit further comprises an antifungal such as a polyene (e.g. amphotericin B), an azole (e.g. thiabendazole, miconazole, fluconazole), and/or an echinocandin (e.g. caspofungin). In some embodiments, the kit may further comprise a lipoprotein such as human low-density lipoproteins (LDL) and/or human high-density lipoproteins (HDL). In some alternative embodiments, the kit further comprises feeder cells that support the growth of HSPCs.

In some embodiments, the components included in the kit are all packaged individually. In other embodiments, some of the components included in the kit are packaged together. In some embodiments, the agonist of the TAM receptor, the ligand of the TAM receptor and/or the compound capable of inducing the expression of the TAM ligand is packaged individually and the SR1, UM171, AA2P and/or VPA are packaged together. The components of the kit may be packaged in, for example, blister packs, vials, ampoules, bottles, bags, boxes, syringes, flasks and beakers.

The present invention will be more readily understood by referring to the following examples which are given to illustrate the invention rather than to limit its scope.

### EXAMPLE

Using statistical design of experiments a series of five Stem Cell Agonist Cocktails (SCACs) were developed: SMA, SM2, SM6, X2A and X2B. The composition of the cocktails are summarized in Table 1.

TABLE 1
Composition of the Stem Cell Agonist Cocktails
(SCACs) and their growth promoting attribute.

| Small molecules | | | | | |
|---|---|---|---|---|---|
| (stem cell agonists) | | | | | |
| | SR1 | UM171 | VPA | AA2P | |
| SCACs | (nM) | (nM) | (mM) | (μM) | SCAC Attributes |
| SMA | 5023 | 0.35 | 0.502 | 1000 | RS model, to maximize CD34+CD45RA− cells |
| SM2 | 5023 | 125.63 | 0.01 | 1000 | RS model, to maximize EPCRHigh cells |
| SM6 | 1000 | 38 | 0.125 | 250 | Derived from SMA, maximizes TNC, |
| | | | | | CD34+ and CD34+CD45RA− cells |
| X2A | 2500 | 62 | 0.01 | 1000 | Derived from SM2, maximizes TNC, CD34+ |
| | | | | | and CD34+CD45RA− and EPCR^{High} cells |
| X2B | 2500 | 62 | 0.01 | 0.1 | Derived from X2A, to address functional |
| | | | | | contribution of AA2P |
| C6 | 750 | 35 | 0 | 0 | Published benchmark control |
| | | | | | (Fares et al., 2014) |

SR1 = StemReginin 1,
UM171 = PubChem CID 71714981,
VPA = valproic acid,
AA2P = L-ascorbic acid 2-phosphate magnesium salt hydrate.
RS: response surface

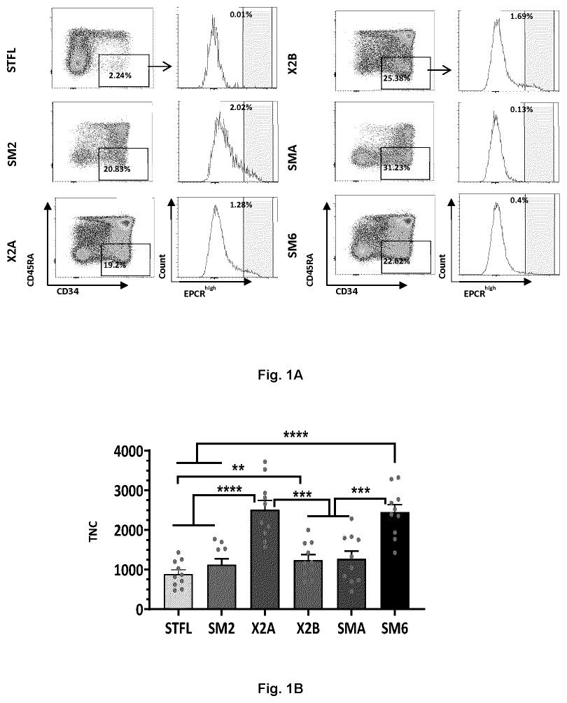

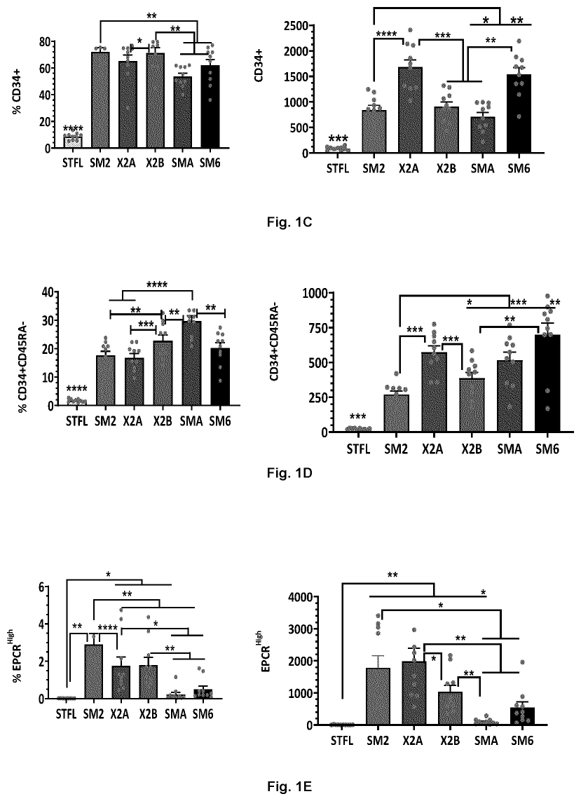

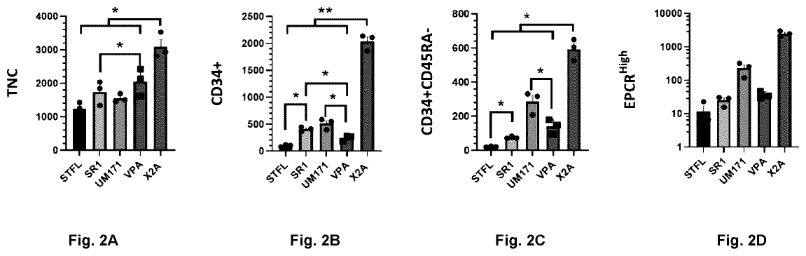

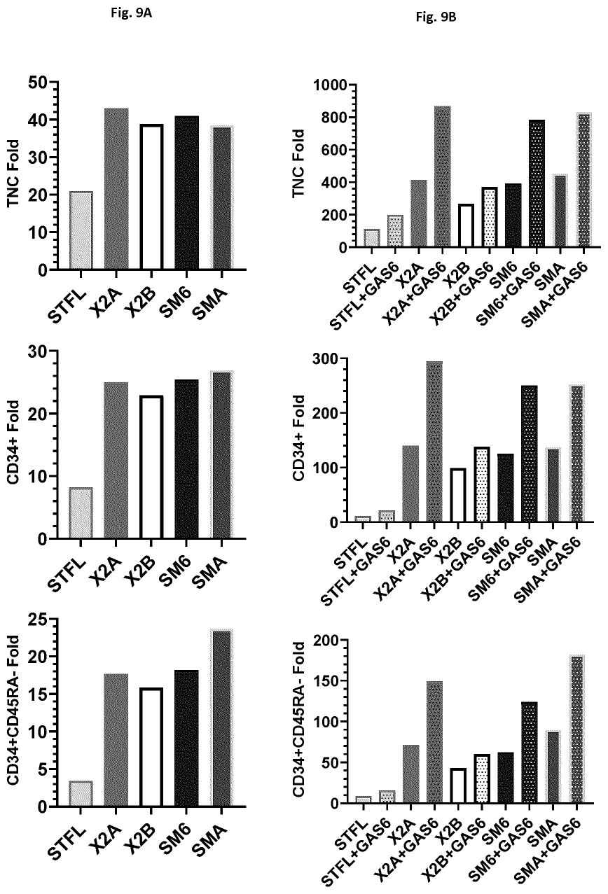

Umbilical cord blood (UCB) CD34+ cells were cultured in StemSpan™ SFEM (StemCell Technologies) in the presence of different SCACs for 14-days and net fold expansion of TNC, CD34+ cells CD34+CD45RA− cells, and EPCR^{High} was calculated based on respective population on day-0 (e.g. net TNC day 14/TNC day 0). Control STFL cultures consisted of the same complete media without small molecules. Unless indicated otherwise, all cultures were supplemented with Stem Cell Factor, Flt-3 and Thrombopoietin at 100 ng/ml. All cultures also contained 10 μg/mL low-density lipoprotein (LDL, Stem Ce, Technologies) and 1% penicillin-streptomycin (Gibco). The capacity of the SCACs to support the growth of HSPC is presented in FIGS. 1A to 1E. In fact, FIGS. 1A to 1E show that the expansion of UCB hematopoietic stem cells and progenitor-enriched (HPSC) subsets is significantly increased by the various Stem Cell Agonist Cocktails (SCACs). The improvement in HSPC expansion provided by the SCACs is exemplified with X2A in FIGS. 2A-2D. FIGS. 2A to 2D show that the expansion of CB TNC and CD34+ subsets were enhanced in the presence of X2A compared to single molecule conditions. Culture done with adult CD34+ mobilized peripheral blood stem cells (mPBSC, CAT #70060.2, Stem Cell Technologies) also contained IL-6 (100 ng/ml) as additional cytokine (FIG. 9).

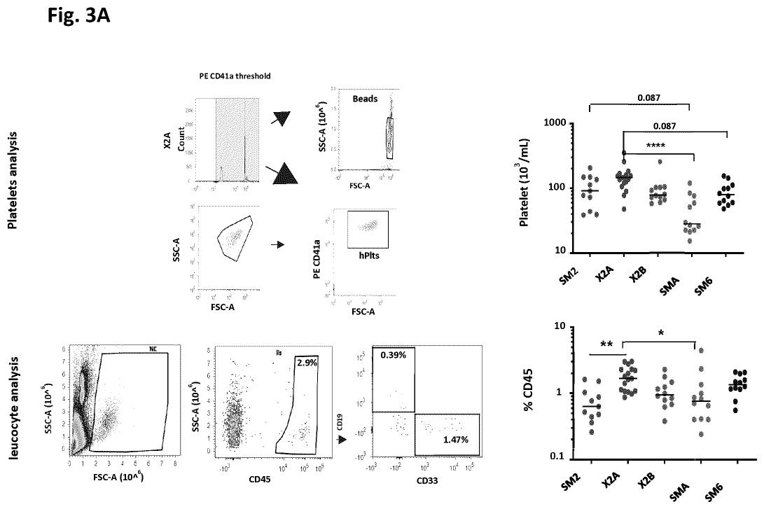

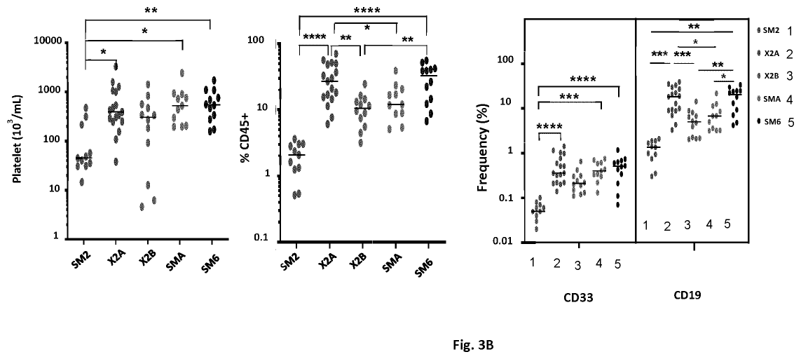

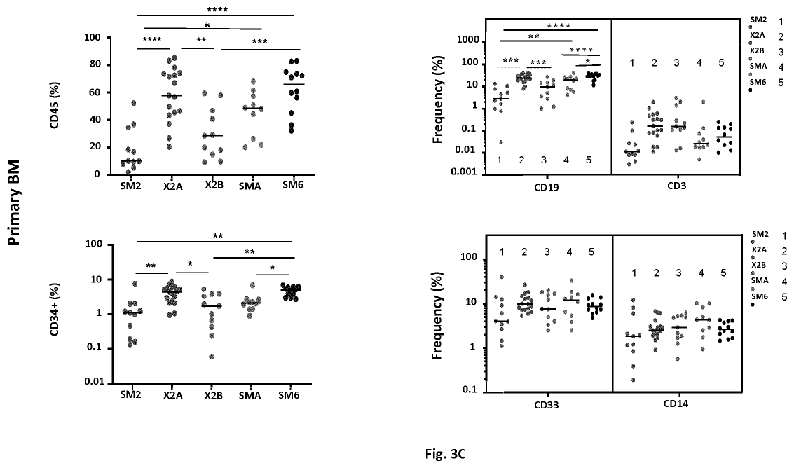

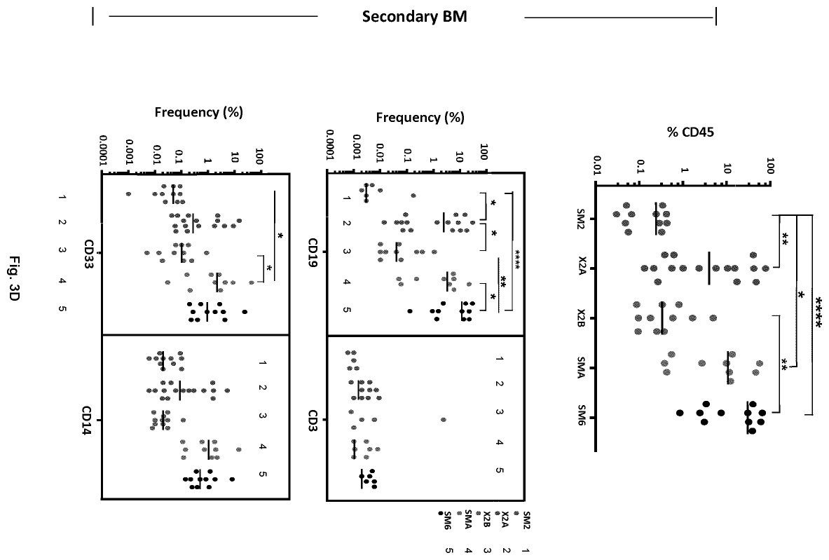

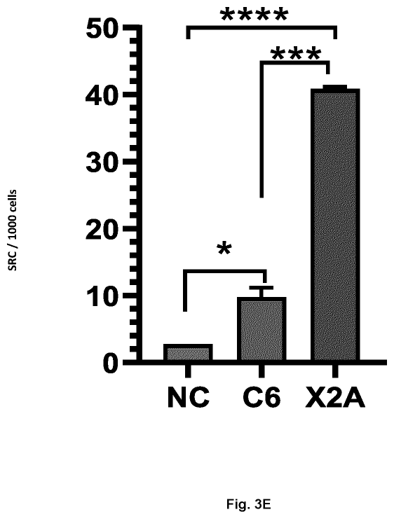

The progeny of 1 250 UCB CD34+ cells cultured in presence of SCACs for 14-days were transplanted i.v. into irradiated (300 cGy) 8 weeks old NOD.Cg-Prkdc^{scid} II2rg^{tm1Wjl}/SzJ (NSG, Jackson Laboratory, Bar Harbor, USA) mice. Chimerism was measured using flow cytometry in peripheral blood at week-3 and -16 post-transplantation for platelets and leucocytes using methods that are known in the art. BM chimerism was measured 18-weeks post-transplantation by flow cytometry analyses of humanized bone marrow cells. FIGS. 3A to 3E show the engraftment properties of SCAC-expanded CB HSC grafts in mice, with X2A and SM6 SCACs providing superior engraftment. It can be seen from FIGS. 3A-3E that short-term (ST) engraftment was superior with X2A-expanded HSPCs. Long-term (LT) platelet and leucocyte engraftment were superior in X2A or SM6 recipients. All SCACs support long-term engraftment and that X2A and SM6 generally provide the best engraftment of all.

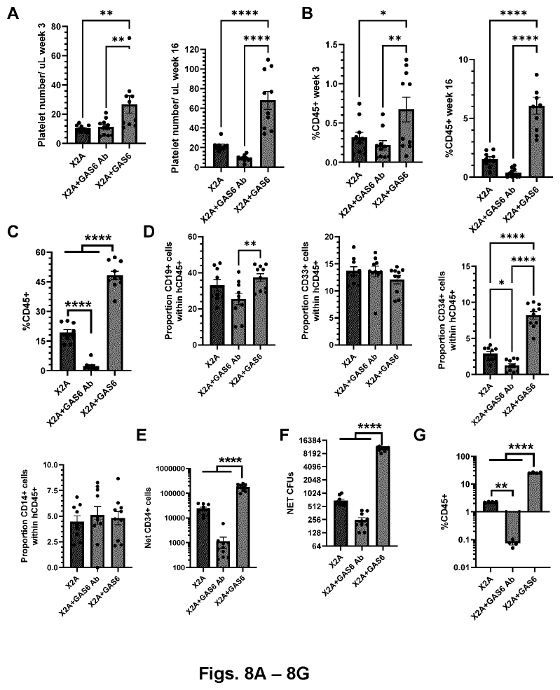

Serial multilineage engraftment analysis: sublethally irradiated secondary NSG recipients were transplanted with 80% of the bone marrow (BM) i.v. collected from individual primary recipients. Engraftment in the BM was analyzed 12 weeks post-transplant. X2A, SM6 and SMA had the best level of human BM engraftment in secondary recipients (FIG. 3D, and FIG. 8G).

FIG. 3E indicates that the SCAC X2A increased the number of scid repopulating cells (SRC) when compared to C6 (control as described in Fares et al., 2014). For LDA transplantation, fresh CD34+ NCs (250, 500, 1000 cells) or the progeny of 25, 100, 250, 500 starting CD34+ cells (day 0 equivalent) cultured for 14-day were transplanted i.v. into irradiated NSG mice. Human BM chimerism was investigated 20-week post-transplantation. Mice were considered positive for human engraftment when human CD45+CD33+ cells were ≥0.10%.

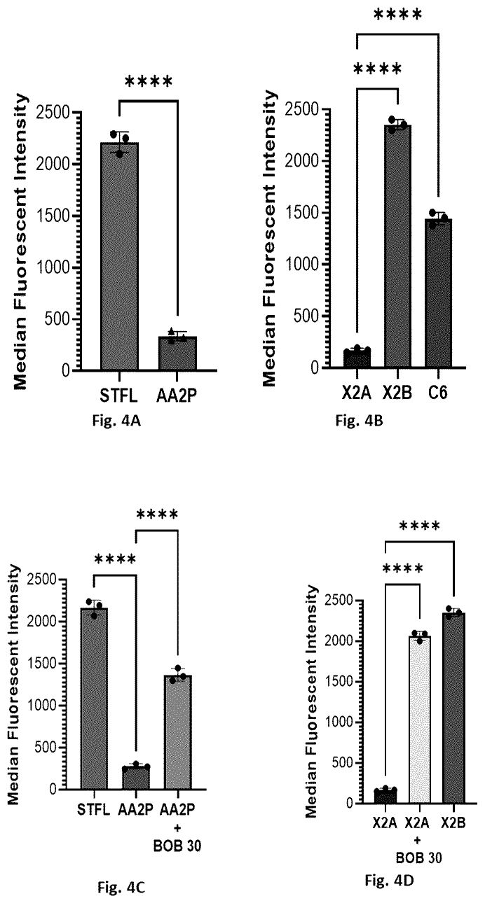

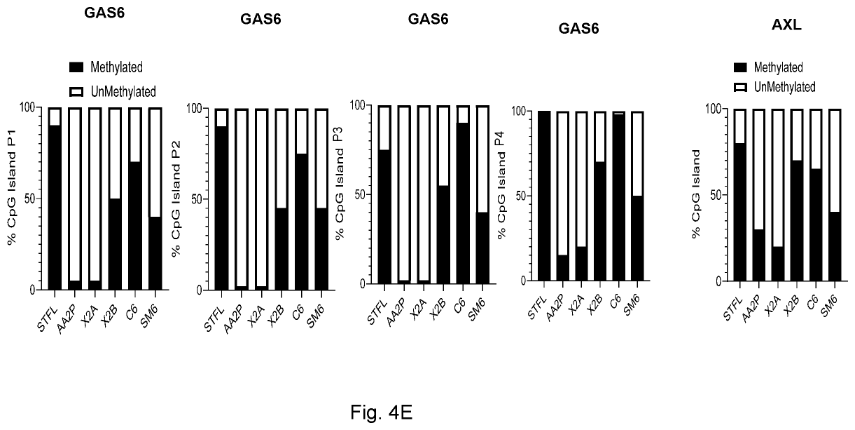

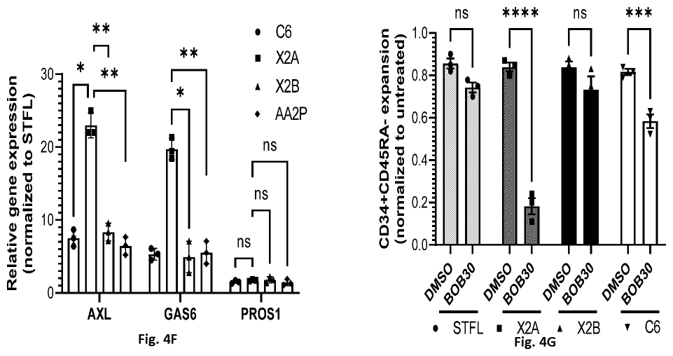

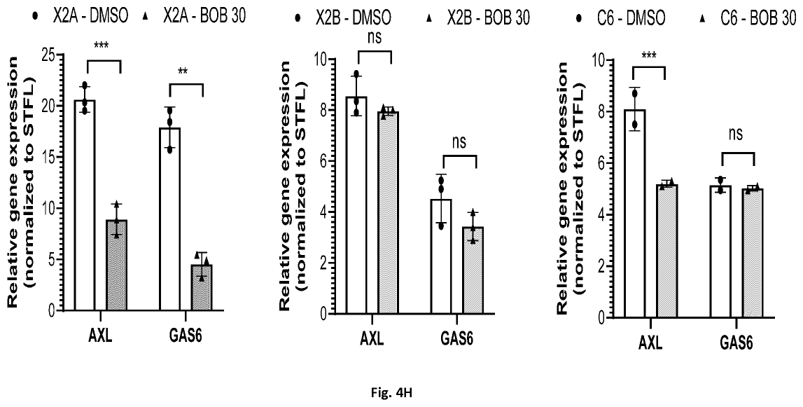

To investigate the impact of AA2P and/or SCACs on DNA methylation level and expression of selected genes, HSPC were cultured in STFL with or without AA2P or indicated SCAC for 14 days (FIGS. 4A-4H). In fact, FIGS. 4A to 4H show that L-ascorbic acid 2-phosphate magnesium salt hydrate (AA2P) promoted DNA demethylation and lead to increased expression of AXL and GAS6 which promoted HSPC expansion in the presence of SCACs or C6. In some cultures, the TET inhibitor bobcat339 was added to cultures at day 10, and the impact on DNA methylation and HSPC expansion was investigated in indicated cultures. The level of DNA methylation was tracked by flow cytometry while expression of indicated genes (or methylation level of indicated CpG islands) were analyzed by qPCR from purified expanded CD34+CD45RA− HSPC. Methylation analyses were performed using the OneStep qMethyl™ Kit (Zymo Research, Cat #D5310) according to manufacturer's instructions. Briefly, 250,000 CD34+45RA− cells cultured in the indicated culture conditions and were sorted on day 7 and day 14 for methylation analyses. DNA was digested according to the manufacturer's instructions and qPCR was performed using the BioRad CFX96™ instrument. The primers used for qPCR targeted the CpG islands near the promoter of our target genes.

The methylation level of cytosine (5-methylcytosine, 5MC) residue in the DNA of cultured HSPC cells was significantly different in AA2P and X2A cultures compared to STFL control, X2B or C6 cultures (FIGS. 4A and 4B). This was associated with increased expression of AXL and GAS6. The expression of GAS6 is greatest in X2A cultures (FIGS. 4F and G). Inhibition of ten-eleven translocation (TET) methylcytosine dioxygenases with BOBCat inhibitor (Bobcat339) increased the methylation in AA2P and X2A cultures (FIGS. 4C and 4D), which reduced the expression of AXL and GAS6 (FIG. 41) leading to reduced cell growth (FIG. 4H). The level of 5 mC was determined by intracellular flow cytometry with anti-5-methylcytosine (clone 33D3, Millipore Sigma, Cat #MABE146) and appropriate secondary antibody.

The level of CpG island DNA methylation in the promoters of the AXL and GAS6 genes were investigated in the indicated culture conditions (FIG. 4E). AA2P and X2A cultures produced CD34+CD45RA− HSPC with the lowest level of methylated CpG islands in AXL and GAS6 genes. C6 cultures had higher level of methylation, while SM6 had methylation levels in between X2A and C6. Hypomethylation of CpG islands in enhancer and promoter of genes is associated with increased gene expression.

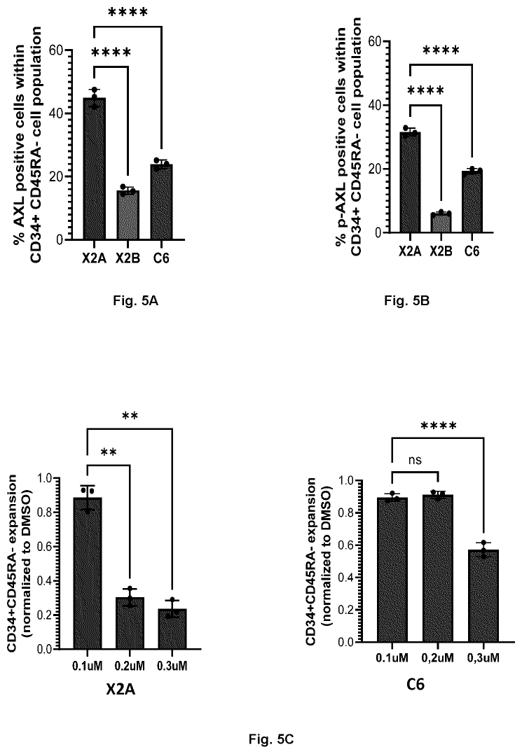

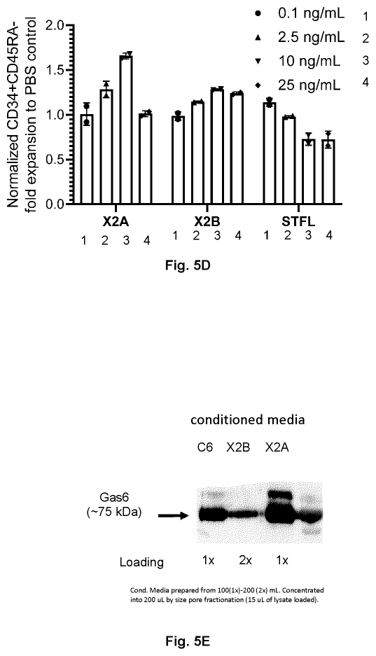

To investigate the impact of AA2P and/or SCACs on expression of AXL and its activation level, HSPC were culture in STFL with or without AA2P or the indicated SCAC for 14 days (FIG. 4F). In some cultures, the AXL kinase inhibitor Bemcentinib or a neutralizing antibody was added to cultures at day 10, and the impact on HSPC expansion was investigated in indicated cultures at day 14. Proteins level were investigated by western blot analysis or intracellular flowcytometry analysis following common procedures with anti-AXL [Goat IgG anti-AXL (Cat #AF154) or anti-p-AXL (Cat #AF2228) or anti-GAS6 (rabbit IgG, Cat #67202S, Cell Signaling Tech.) and appropriate secondary antibodies, R&D systems). FIGS. 5A to 5G show that the activation of AXL on HSPC in SCAC cultures promoted robust expansion of CB HSPCs and supplementation of GAS6 enhanced the expansion of HSPCs in SCAC cultures. AXL is expressed by CD34+CD45RA− HSPCs cultured in both X2A and C6 culture (FIG. 5A). However, the amount of activated AXL (pAXL) is significantly higher within X2A cultured CD34+CD45RA− HSPCs (FIG. 5B). Inhibition of AXL kinase activity using a chemical inhibitor (Bemcentinib) resulted in a reduction in the ability of X2A and C6 to expand HSPCs (FIG. 5C). GAS6 present in HSPC culture improved HSPC expansion when cultured in the presence of X2A, X2B, STFL (FIGS. 5D and 5E) or SM6 (FIG. 5F). Notably, Axl and GAS6 expression were both significantly increased in X2A compared to X2B and C6 (FIGS. 4F, 5A, 5B and 5E). This support that, even in the absence of exogenous GAS6 , AA2P alone or in combination with other small molecule (i.e. stem cell agonist such as UM171, SR1 . . . ) is capable of driving enough increased endogenous expression of GAS6 and AXL to support the improved expansion of CD34+CD45RA− HSPCs through para-and/or autocrine activation of AXL on HSPCs. Addition of exogenous GAS6 to SCAC and C6 cultures further improved HSPC expansion compared to treatment with SCACs and C6 alone (FIGS. 5F-5G). Addition of GAS6 neutralizing antibody that blocks GAS6 binding to AXL and AXL activation, results in reduction in ability of X2A, X2B, C6 and SM6 to expand HSPC cells (FIG. 5G).

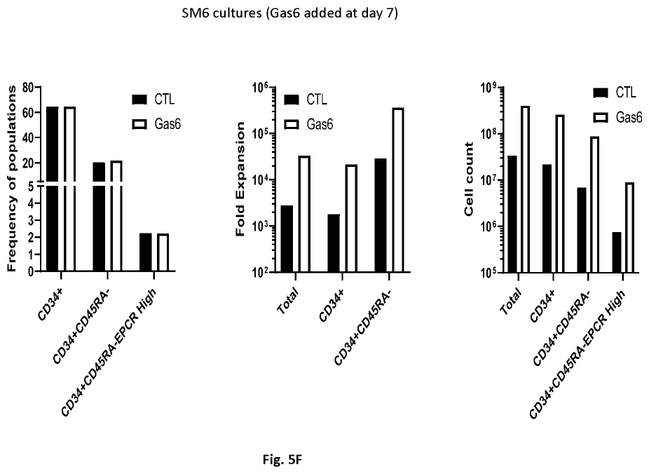

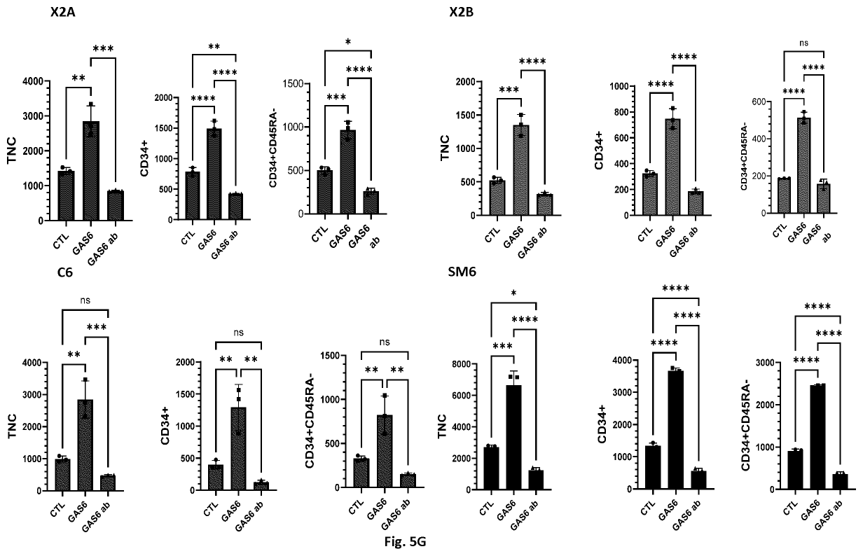

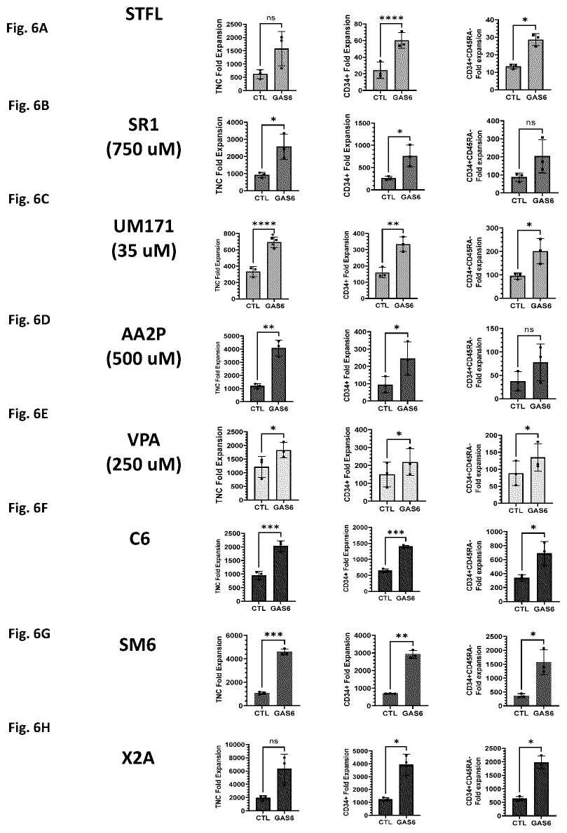

FIGS. 6A to 6H show that the activation of AXL by GAS6, in combination with different single stem cell agonists or agonist cocktails in HSPC cultures strongly increases the expansion of the CB HSPC population. In each case, GAS6 was added to the cultures from days 7 to 14. Indeed, as shown in therein, addition of GAS6 to control STFL cultures (6A) or to previously developed single stem cell agonist-based (e.g. SR1 (6B), UM171 (6C), AA2P (6D), VPA (6E), C6 (6F), SM6 (6G) or X2A (6H) based expansion platforms strongly increase the expansion of TNC and HSPC subsets, demonstrating that GAS6 can complement many HSPC expansion platforms. HSPC were cultured as indicated for 14 days with addition of GAS6 (10 ng/mL) from day 7 to day 14.

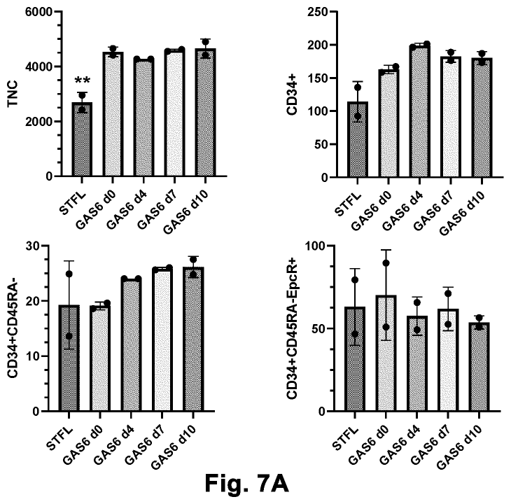

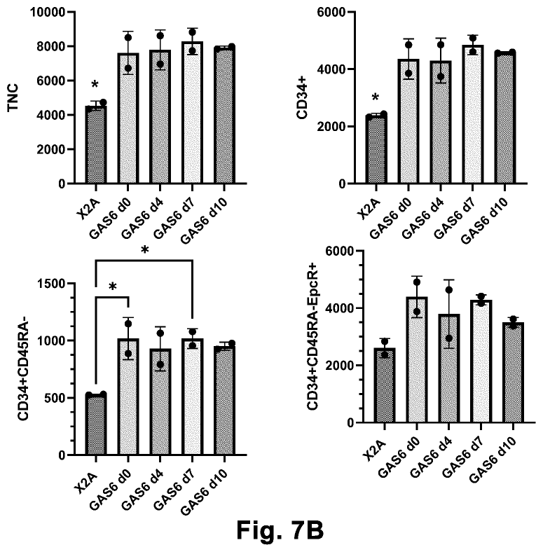

FIGS. 7A-7B show that the activation of AXL by GAS6 supplementation at different time point in culture leads to superior expansion of CB total nucleated cells (TNC) and HSPC subsets in STFL cultures supplemented or not with the SCAC X2A. Cell expansion measured after 14 days of culture in STFL culture (FIG. 7A) and in X2A cultures (FIG. 7B). Indeed, as shown therein, the rise in cell growth (TNC) and expansion of HSPC subsets (CD34+ cells, CD34+CD45RA−, EPCRHigh cells) induced by GAS6 can be accomplished by adding GAS6 in cultures at various time point, as early as day 0 and as late as day 10, preferably at day 7.

FIGS. 8A-8G shows that the activation of AXL by GAS6 supplementation in X2A UCB HSPC cultures significantly increase the serial engraftment achieved from the expanded HSPC. Mice group were transplanted with the progeny of 750 expanded CD34+ cells after 14 days of culture. The groups were; X2A-expanded cells, X2A-expanded cells supplemented with GAS6 (10 ng/ml) or a GAS6 neutralizing antibody (GAS6 Ab) from day-10 to day-14. As shown therein, addition of GAS6 to CB HSPC expansion cultures improves the engraftment achieved from the ex vivo expanded HSPCs. As exemplified in FIGS. 8A-8B, levels of human platelets and human % CD45+ leucocytes were significantly superior in mice transplanted with HSPC produced in X2A cultures supplemented with GAS6 (X2A+ GAS6 group) at 3-weeks and 16-weeks post-transplant. This result demonstrates that GAS6 supplementation improves both short-and long-term engraftment. As shown in FIG. 8C, addition of GAS6 also improved long-term human BM engraftment (% CD45+). In contrast, blocking AXL activation using a GAS6 neutralizing antibody (X2A+GAS6 Ab group) had the opposite effect on engraftment (FIGS. 8A-8C). Importantly, engraftment in X2A+ GAS6 group showed normal distribution of human lineages downstream HSC, showing normal differentiation (FIG. 8D). However, the proportion of CD34+ HSPC was found significantly superior in X2A+ GAS6 mice suggesting increased levels of human HSPC in these mice. Consistent with this, the net number of human CD34+ BM cells (FIG. 8E) and human CFU (FIG. 8F) were significantly superior in X2A+ GAS6 primary transplants. Finally, secondary transplants demonstrated that primary X2A+ GAS6 grafts had superior serial engraftment activity than X2A grafts (FIG. 8G). This results demonstrate increased HSC activity in expansion X2A+ GAS6 HSPC cultures.

FIGS. 9A-9B show that the expansion of HSPC-enriched subsets is significantly increased by the various SCACs in adult mobilized peripheral blood stem cell (mPBSC) cultures. It also shows that the activation of AXL by GAS6 supplementation in mPBSC cultures raises cell growth and HSPC expansions. In each case, GAS6 was added to the cultures from days 7 to 14. As shown therein, the addition of SCAC to CD34+ mPBSC STFL cultures significantly improve expansion of TNC and CD34+ and CD34+CD45RA− HSPC subsets after 7 (FIG. 9A) and 14 (FIG. 9B) days of culture when compared to the STFL control cultures. Also, addition of GAS6 to the indicated cultures from day 7 to 14 further increased the expansion of both TNC and HSPC subsets in STFL control culture and in SCAC-cultures as previously seen for UCB derived HSPCs (FIG. 9B).

The expanded population of normal HSPCs obtained by the in vitro methods described herein do, however, differ in some respects compared to normal HSPCs. For example, it was found that SCAC-expanded HSPCs have altered epigenetic marks (e.g., loss of H3K27me3 or H3K27me2 methylation compared to controls). It was also found that approximately 9000 genes were differentially expressed in SCAC-expanded populations of normal HSPCs obtained using the in vitro methods described herein compared to controls. Notably, among the 9000 differentially expressed genes identified, AXL and GAS6 were both found to be upregulated in the expanded HSPC population compared to controls. Tables 2-5 shows the genes identified by next generation gene sequencing that are up or down regulated in CD34+CD45RA− cells by either SM6 or X2A when compared to non-cultured CD34+CD45RA− counterpart.

Lengthy table referenced here
US20240368549A1-20241107-T00001
Please refer to the end of the specification for access instructions.

Lengthy table referenced here
US20240368549A1-20241107-T00002
Please refer to the end of the specification for access instructions.

Lengthy table referenced here
US20240368549A1-20241107-T00003
Please refer to the end of the specification for access instructions.

Lengthy table referenced here
US20240368549A1-20241107-T00004
Please refer to the end of the specification for access instructions.

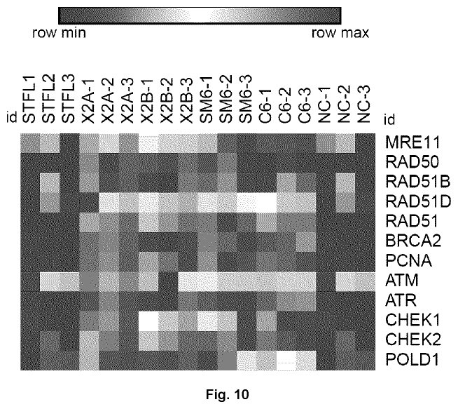

Further, as can be seen in FIG. 10, several different classes of DNA repair enzyme genes are upregulated in SCAC cultures (e.g. X2A) when compared to non-cultured (NC) cells or cells cultured in STFL culture. Expression of these genes amongst others can facilitate error free gene editing through homology directed repair. In addition, the presence of AA2P in SCAC culture hypomethylates the DNA within HSPCs, making the DNA more accessible to gene editing complexes containing CRISPR-Cas proteins, TALENs and Zinc fingers.

While the disclosure has been described in connection with specific embodiments thereof, it will be understood that the scope of the claims should not be limited by the preferred embodiments set forth in the examples, but should be given the broadest interpretation consistent with the description as a whole.

### REFERENCES

Boitano A. E., et al. (2010). “Aryl hydrocarbon receptor antagonists promote the expansion of human hematopoietic stem cells.” Science 329 (5997): 1345-1348
Chaurasia P., et al. (2014). “Epigenetic reprogramming induces the expansion of cord blood stem cells.” J Clin Invest 124 (6): 2378-2395.
Agathocleous M., et al. (2017). “Ascorbate regulates haematopoietic stem cell function and leukaemogenesis.” Nature 549 (7673): 476-481.2378-2395.
Fares I., et al. Pyrimidoindole derivatives are agonists of human hematopoietic stem cell self-renewal. Science. 2014 Sep. 19; 345 (6203): 1509-12. doi: 10.1126/science. 1256337. PMID: 25237102; PMCID: PMC4372335.
Manesia J. et al., “Stringent small molecule dose requirements for the optimal expansion of hematopoietic stem cells revealed by predictive analytics and xenotransplants” in 61st American Society of Hemtology (ASH) Annual Meeting and Exposition held from Dec. 7-10, 2019, Orlando, Florida (poster presentation and conference abstract).
Manesia J. et al., “Systemic Optimization of Small Molecule Cocktail to Enhance Hematopoietic Stem Cells Expansion”, in the 2019 American Association of Blood Banks (AABB) Annual Meeting held from Oct. 19-22, 2019, San Antonio, Texas (poster presentation and conference abstract).
Manesia J. et al., “Development of a small molecule-based hematopoietic stem and progenitor expansion protocol to accelerate engraftment” presented at the 2018 Canadian Bone marrow Transplant Group Annual Meeting held from Jun. 6-9, 2018, Ottawa, Ontario (PowerPoint Presentation).

LENGTHY TABLES
The patent application contains a lengthy table section. A copy of the table is available in electronic form from the USPTO web site (https://seqdata.uspto.gov/?pageRequest=docDetail&DocID=US20240368549A1). An electronic copy of the table will also be available from the USPTO upon request and payment of the fee set forth in 37 CFR 1.19(b)(3).

1. An in vitro method of expanding normal hematopoietic stem and progenitor cells (HSPCs), the method comprising contacting one or more ligand of an AXL receptor comprising a growth arrest 6 (GAS6) polypeptide, a variant of the GAS6 polypeptide having ligand activity towards the AXL receptor or a fragment of the GAS6 polypeptide having ligand activity towards the AXL receptor with the normal HSPCs in a culture medium to provide an expanded population of normal HSPCs, wherein the GAS6 polypeptide, the variant of the GAS6 polypeptide having ligand activity toward the AXL receptor or the fragment of the GAS6 polypeptide having ligand activity towards the AXL receptor is present in the culture medium at a concentration of between about 0.1 ng/ml to about 100 ng/ml.

2. The in vitro method of claim 1, wherein the normal HSPCs comprise human cells.

3. The in vitro method of claim 1, wherein the normal HSPCs are derived from cord blood, placenta, bone marrow, peripheral blood, embryonic tissue, induced pluripotent stem cells (iPSCs), or fetal tissue.

4. The in vitro method of claim 1, wherein the one or more ligand of the AXL receptor comprising a growth arrest 6 (GAS6) polypeptide, the variant of the GAS6 polypeptide having ligand activity towards the AXL receptor or the fragment of the GAS6 polypeptide having ligand activity towards the AXL receptor, after having contacted the normal HSPCs, is capable of improving the expansion of the normal HSPCs, when compared to control cells.

5. The in vitro method of claim 1 further comprising contacting the normal HSPCs with a stem cell agonist cocktail comprising StemReginin1, UM171, L-ascorbic acid 2-phosphate magnesium salt hydrate (AA2P) and/or valproic acid (VPA).

6. The in vitro method of claim 5, wherein the normal HSPCs are contacted with the stem cell agonist cocktail prior to contacting the one or more ligand of the AXL receptor comprising a growth arrest 6 (GAS6) polypeptide, the variant of the GAS6 polypeptide having ligand activity towards the AXL receptor or the fragment of the GAS6 polypeptide having ligand activity towards the AXL receptor.

7. The in vitro method of claim 5, wherein the stem cell agonist cocktail comprises:
a) about 100 nM to about 5025 nM of StemReginin1;
b) about 0.10 nM to about 150 nM of UM171;
c) about 0.1 μM to about 2 000 μM of AA2P; and/or
d) about 0.01 mM to about 1 mM of valproic acid.

8. The in vitro method of claim 1, wherein the expanded population of normal HSPCs comprise cells:
a) expressing the surface proteins CD34, CD90 and/or CD49f on their cell membrane;
b) failing to express or expressing a lower amount of the surface protein CD45RA on their cell membrane compared to the HSPCs; and/or
c) expressing a higher amount of the surface protein EPCR on their cell membrane compared to the HSPCs.

9. The in vitro method of claim 1 comprising culturing the normal HSPCs in the absence of feeder cells.

10. The in vitro method of claim 5, wherein the normal HSPCs are cultured:
a) in medium supplemented with one or more cytokine;
b) in the presence of the stem cell agonist cocktail; and/or
c) in the presence of the GAS6 polypeptide, the variant of the GAS6 polypeptide or the fragment of the GAS6 polypeptide for at least 2 days.

11. An expanded population of normal HSPCs obtainable or obtained by the process of claim 1.

12. A method for treating a condition in a subject in need thereof comprising:
a) providing the expanded population of normal HSPCs of claim 11; and
b) grafting the expanded population of normal HSPCs to the subject to treat the condition.

13. The method of claim 12 further comprising obtaining the normal HSPCs used to provide the expanded population of normal HSPCs from the subject.

14. The method of claim 12, wherein the condition being treated comprises:
a) a cancer;
b) a neural disorder;
c) an immune deficiency;
d) an auto-immune disorder;
e) a metabolic disorder; and/or
f) a genetic disorder.

15. The method of claim 12, wherein the subject is a human.

16. The method of claim 15, wherein the subject is an adult.

17. The method of claim 15, wherein the subject is a child.

18. A kit for the expansion of normal HSPCs in a culture medium, the kit comprises one or more ligand of an AXL receptor comprising a growth arrest 6 (GAS6) polypeptide, a variant of the GAS6 polypeptide having ligand activity towards the AXL receptor or a fragment of the GAS6 polypeptide having ligand activity towards the AXL receptor, wherein the GAS6 polypeptide, the variant of the GAS6 polypeptide having ligand activity toward the AXL receptor or the fragment of the GAS6 polypeptide having ligand activity towards the AXL receptor is present in the culture medium at a concentration of between about 0.1 ng/ml to about 100 ng/ml together with at least one of the following components:
StemReginin 1;
UM171;
L-Ascorbic acid 2-phosphate magnesium salt hydrate (AA2P); and/or
valproic acid.

19. (canceled)

20. The kit of claim 18, further comprising:
a cell culture medium;
albumin;
a buffer;
a vitamin;
an amino acid;
a cytokine;
a mineral or trace element;
serum or a serum substitute; and/or
a lipid.

21. The kit of claims 18, further comprising:
an antibiotic;
an antifungal; and/or
a lipoprotein.

22. (canceled)

23. (canceled)

24. The kit of claim 18 further comprising feeder cells on which the normal HSPCs can be cultured.
